(19) United States
(12) Patent Application Publication
(10) Pub. No.: US 2009/0155349 A1
(43) Pub. Date: Jun. 18, 2009

(54) METHODS OF TREATING INFLUENZA VIRAL INFECTIONS

(76) Inventors: Jonathan Daniel Heller, San Francisco, CA (US); Scott Matthew Laster, Cary, NC (US); Rocio Alejandra Lopez, Raleigh, NC (US); Neil Frazer, Cary, NC (US)

Correspondence Address: DARBY & DARBY P.C., P.O. BOX 770, Church Street Station, New York, NY, 10008-0770, US

(21) Appl. No.: 12/280,463

(22) PCT Filed: Feb. 23, 2007
(86) PCT No.: PCT/US07/62730

§ 371 Date: Aug. 22, 2008

Related U.S. Application Data
(60) Provisional application No. 60/776,043, filed on Feb. 23, 2006.
(60) Provisional application No. 60/775,869, filed on Feb. 23, 2006.

(51) Int. Cl. A61K 9/127; A61K 31/05; A61K 31/136; A61K 31/225; A61K 31/24; A61K 31/195; A61K 31/351; A61K 31/405; C12N 5/08; C12N 7/06; A61K 9/14; A61P 31/16

(52) U.S. Cl. 424/450; 514/734; 514/659; 514/548; 514/537; 514/563; 514/459; 514/419; 435/375; 435/238; 424/501

(57) ABSTRACT

Methods are described for treating an influenza viral infection or associated diseases, disorders or mechanisms in a subject, comprising administering to the subject a therapeutically effective amount of a catecholic butane of the general formula (I) or a pharmaceutically acceptable salt thereof:

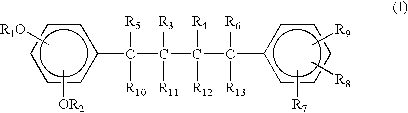

wherein R₁ and R₂ each independently represents a hydrogen, a lower alkyl, a lower acyl, an alkylene, or —OR₁ and —OR₂ each independently represents an unsubstituted or substituted amino acid residue or pharmaceutically acceptable salt thereof; R₃, R₄, R₅, R₆, R₁₀, R₁₁, R₁₂ and R₁₃ each independently represents a hydrogen, or a lower alkyl; and R₇, R₈ and R₉ each independently represents a hydrogen, —OH, a lower alkoxy, a lower acyloxy, an unsubstituted or substituted amino acid residue or pharmaceutically acceptable salt thereof, or any two adjacent groups together may be an alkyene dioxy; with the proviso in certain circumstances that where one of R₇, R₈ and R₉ represents a hydrogen, then —OR₁, —OR₂ and the other two of R₇, R₈ and R₉ do not simultaneously represent —OH.

### CROSS-REFERENCE TO RELATED APPLICATIONS

This application claims the benefit of U.S. Provisional Application No. 60/775,869, filed Feb. 23, 2006, and U.S. Provisional Application No. 60/776,043, filed Feb. 23, 2006, the disclosures of which are hereby incorporated herein by reference in their entireties.

### BACKGROUND OF THE INVENTION

Influenza viruses are prevalent sources of infection in a variety of species and cause severe cold-like symptoms and can often lead to respiratory disorders and/or lethal pneumonia. Influenza viruses are classified into three types, namely types A, B, and C, on the basis of differences in the serotypes of nucleoproteins and membrane proteins. Of these, influenza virus type A and influenza virus type B are prevalent every year. The influenza type A viruses have two glycoproteins, i.e., a hemaglutinin (HA) and a neuraminidase (NA), on the surface of an envelope thereof and are thus classified into subtypes on this basis, such as H1N1, H2N2 and H3N2 on the basis of the antigenecities of the proteins. Influenza type B and influenza type C each have only one subtype.

Influenza type A viruses undergo substantial changes in antigenecity and prevail every year above other types of influenza. Antiviral agents for influenza type A viruses are known, but are not wholly satisfactory because they often cannot cope with mutations of the virus. The inability of antiviral agents to cope with the mutations of the virus is most likely due to the severity of antigenic variations of the virus.

All type A influenza viruses, including those that regularly cause seasonal epidemics of influenza in humans, are genetically labile and well-adapted to elude host defenses. Influenza viruses lack mechanisms for “proofreading” and repair of errors that occur during replication. As a result of these uncorrected errors, the genetic composition of the viruses changes as they replicate in humans and animals, and the existing strain is replaced with a new antigenic variant. These constant, permanent and usually small changes in the antigenic composition of influenza A viruses are known as antigenic “drift”.

The tendency of influenza viruses to undergo frequent and permanent antigenic changes necessitates constant monitoring of the global influenza situation and annual adjustments in the composition of influenza vaccines.

Influenza viruses have an additional characteristic of great public health concern. Namely, influenza type A viruses, including subtypes from different species, can swap or reassort generic materials and merge. This reassortment process, known as antigenic shift, results in novel subtypes of the virus different from both parent viruses. As populations will have no immunity to the new subtype, and as no existing vaccines can confer protection, antigenic shift has historically resulted in highly lethal pandemics of influenza. For this to happen, the novel subtype needs to have genes from human influenza viruses that make it readily transmissible from person to person for a sustainable period.

Conditions favorable for the emergence of antigenic shift have often been thought to involve humans living in close proximity of other domesticated species infected with various strain of influenza viruses. Pigs, for example, are susceptible to infection with both avian and mammalian viruses, including human strains. Thus, they can serve as a “mixing vessel” for the scrambling of genetic material from human and avian viruses, resulting in the emergence of a novel subtype. However, recently, another possible mechanism has been identified for the emergence of antigenic shift. It has been suggested that humans themselves can serve as a mixing vessel for the emergence of novel influenza subtypes.

There are currently fifteen (15) known avian influenza virus subtypes. Subtype H5N1 is of particular concern for several reasons. H5N1 mutates rapidly and has a documented propensity to acquire genes from viruses infecting other animal species. Its ability to cause severe disease in humans has been documented on two occasions in Hong Kong in 1997 and 2003. Since then, as of Dec. 14, 2005, the World Health Organization has laboratory-confirmed 138 cases of human infection with H5N1 avian influenza. Of these 138 cases, 71 have been fatal.

In addition, laboratory studies have demonstrated that isolates from this virus have a high pathogenicity and can cause severe disease in humans. Additionally, birds that survive infection with avian influenza subtype H5N1 excrete the virus for at least ten (10) days, thus facilitating further spread at live poultry markets and in migratory birds. The spread of infection in birds increases the opportunities for direct infection of humans. If more humans become infected over time, the likelihood increases that humans, if concurrently infected with human and avian influenza strains, could serve as the mixing vessel for the emergence of a novel subtype with sufficient human genes to be easily transmitted from person to person. This would mark the beginning of an influenza pandemic. Historically, influenza pandemics can be expected to occur, on average, three to four times each century, when new virus subtypes emerge and are readily transmitted from person to person. The occurrence of influenza pandemics is unpredictable. Most influenza experts agree that another influenza pandemic is inevitable and possibly imminent.

While experience in the production of influenza vaccines is considerable, particularly as vaccine compositions change each year to match changes in circulating viruses due to antigenic drift, at least four months are likely necessary to produce any new vaccine in any significant quantities capable of conferring protection against a new virus subtype.

Accordingly, compositions for the treatment of the symptoms of influenza viral infection are often administered to those infected. Given the increased pathogenicity of newer strains of influenza virus, the treatment or mitigation of influenza viral infection symptoms is of increasing importance.

While various treatments for the symptoms of influenza viral infections exist, many are not always effective against newer subtypes including avian strains, and many cause detrimental side effects. Thus, a need exists in the art for new and more effective methods of treating influenza viral infection. The present invention satisfies this need.

### BRIEF SUMMARY OF THE INVENTION

The present invention relates to methods of treating influenza viral infections by the administration of a catecholic butane or a pharmaceutically acceptable salt thereof. While not wishing to be bound by any particular theory, it is believed that the methods of the present invention act to both decrease replication or growth of influenza virus in a host and additionally decrease the occurrence and/or severity of various diseases or disorders accompanying influenza viral infection.

One embodiment of the present invention includes a method of treating an influenza viral infection in a subject. The method comprises administering to the subject a therapeutically effective amount of a catecholic butane of the general formula (I) or a pharmaceutically acceptable salt thereof:

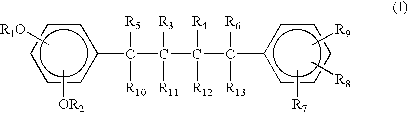

wherein R₁ and R₂ each independently represents a hydrogen, a lower alkyl, a lower acyl, or an alkylene, or —OR₁ and —OR₂ each independently represents an unsubstituted or substituted amino acid residue or pharmaceutically acceptable salt thereof; R₃, R₄, R₅, R₆, R₁₀, R₁₁, R₁₂ and R₁₃ each independently represents a hydrogen, or a lower alkyl; and R₇, R₈ and R₉ each independently represents a hydrogen, —OH, a lower alkoxy, a lower acyloxy, an unsubstituted or substituted amino acid residue or salt thereof, or any two adjacent groups together may be an alkyene dioxy; with the proviso that where one of R₇, R₈ and R₉ represents a hydrogen, then —OR₁, —OR₂ and the other two of R₇, R₈ and R₉ do not simultaneously represent —OH. Substituted or unsubstituted amino acid residues and pharmaceutically acceptable salts thereof are preferably bonded to the aromatic ring at their carboxy terminus.

Another embodiment of the present invention includes a method of treating an influenza viral infection in a subject. The method comprises administering to the subject a therapeutically effective amount of a nordihydroguaiaretic acid derivative of the general formula (II) or a pharmaceutically acceptable salt thereof:

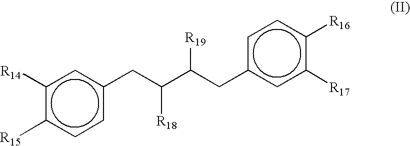

wherein R₁₄, R₁₅, R₁₆ and R₁₇ each independently represents —OH, —OCH₃, —O(C═O)CH₃, or an unsubstituted or substituted amino acid residue or pharmaceutically acceptable salt thereof; R₁₈ and R₁₉ each independently represents —H or a lower alkyl; with the proviso that R₁₄, R₁₅, R₁₆ and R₁₇ are not simultaneously —OH. Substituted or unsubstituted amino acid residues and pharmaceutically acceptable salts thereof are preferably bonded to the aromatic ring at their carboxy terminus.

Another embodiment of the present invention includes a method of treating an avian influenza viral infection in a subject. The method comprises administering to the subject a therapeutically effective amount of a nordihydroguaiaretic acid (NDGA) derivative of the general formula (III) or a pharmaceutically acceptable salt thereof:

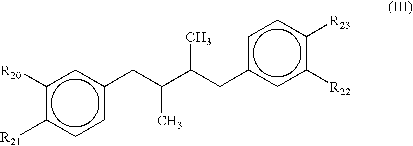

wherein R₂₀, R₂₁, R₂₂ and R₂₃ each independently represents —OH, —OCH₃, —O(C═O)CH₃, or an unsubstituted or substituted amino acid residue or pharmaceutically acceptable salt thereof, with the proviso that R₂₀, R₂₁, R₂₂ and R₂₃ are not simultaneously —OH. Substituted or unsubstituted amino acid residues and pharmaceutically acceptable salts thereof are preferably bonded to the aromatic ring at their carboxy terminus.

Another embodiment of the present invention includes a method of treating an influenza viral infection in a subject. The method comprises administering to the subject a therapeutically effective amount of a composition comprising a catecholic butane selected from the group consisting of tri-O-methyl nordihydroguaiaretic acid (NDGA), tetra-O-methyl NDGA, tetra-glycinyl NDGA, tetra-dimethylglycinyl NDGA, or a pharmaceutically acceptable salt thereof, and a pharmaceutically acceptable carrier or excipient.

Another embodiment of the present invention includes a method of treating a subtype H5N1 influenza viral infection in a human subject. The method comprises orally administering to the human subject a nordihydroguaiaretic acid derivative of the general formula (III) or a pharmaceutically acceptable salt thereof, in an amount of about 10 mg/kg to about 375 mg/kg per dose;

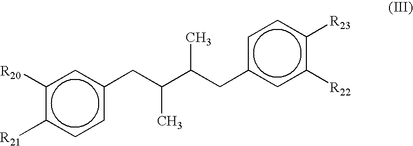

wherein R₂₀, R₂₁, R₂₂ and R₂₃ each represents —OCH₃.

Another embodiment of the present invention includes a method of inhibiting the induction of a proinflammatory cytokine in a cell by an influenza viral infection. The method comprises administering to the cell an effective amount of a catecholic butane of the general formula I or a pharmaceutically acceptable salt thereof:

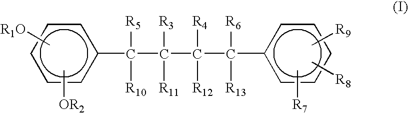

wherein R₁ and R₂ each independently represents a hydrogen, a lower alkyl, a lower acyl, an alkylene, or —OR₁ and —OR₂ each independently represents an unsubstituted or substituted amino acid residue or pharmaceutically acceptable salt thereof; R₃, R₄, R₅, R₆, R₁₀, R₁₁, R₁₂ and R₁₃ each independently represents a hydrogen, or a lower alkyl; and R₇, R₈ and R₉ each independently represents a hydrogen, —OH, a lower alkoxy, a lower acyloxy, an unsubstituted or substituted amino acid residue or salt thereof, or any two adjacent groups together may be an alkyene dioxy.

Another embodiment of the present invention includes a method of inhibiting the induction of a proinflammatory lipid mediator in a cell by an influenza viral infection. The method comprises administering to the cell an effective amount of a catecholic butane of the general formula I or a pharmaceutically acceptable salt thereof:

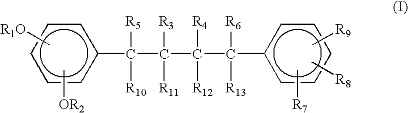

wherein R₁ and R₂ each independently represents a hydrogen, a lower alkyl, a lower acyl, an alkylene, or —OR₁ and —OR₂ each independently represents an unsubstituted or substituted amino acid residue or pharmaceutically acceptable salt thereof; R₃, R₄, R₅, R₆, R₁₀, R₁₁, R₁₂ and R₁₃ each independently represents a hydrogen, or a lower alkyl; and R₇, R₈ and R₉ each independently represents a hydrogen, —OH, a lower alkoxy, a lower acyloxy, an unsubstituted or substituted amino acid residue or salt thereof, or any two adjacent groups together may be an alkyene dioxy.

Another embodiment of the present invention includes a method of inhibiting the induction of tumor necrosis factor alpha (TNF-α) in a macrophage cell by a subtype H5N1 influenza viral infection. The method comprises administering to the macrophage cell an effective amount of a nordihydroguaiaretic acid derivative of the general formula (III) or pharmaceutically acceptable salt thereof:

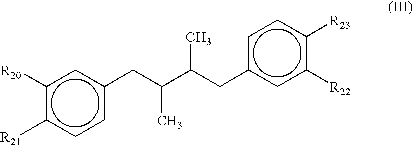

wherein R₂₀, R₂₁, R₂₂ and R₂₃ each represents —OCH₃.

Yet another embodiment of the invention includes a method of inhibiting the induction of prostaglandin E₂ (PGE₂) in a macrophage cell by a subtype H5N1 influenza viral infection. The method comprises the step of administering to the macrophage cell an effective amount of a nordihydroguaiaretic acid derivative of the general formula (III) or pharmaceutically acceptable salt thereof:

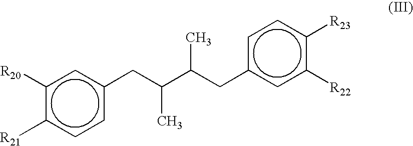

wherein R₂₀, R₂₁, R₂₂ and R₂₃ each represents —OCH₃.

Another embodiment of the invention includes a kit comprising a catecholic butane of the general formula I or a pharmaceutically acceptable salt thereof, and instructions for treating an influenza viral infection in a subject by using the catecholic butane or the pharmaceutically acceptable salt thereof.

Other aspects, features and advantages of the invention will be apparent from the following disclosure, including the detailed description of the invention and its preferred embodiments and the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a graphical representation of lipopolysaccharide (LPS)-induced production of TNF-α by RAW264.7 murine macrophages over time under various conditions.

FIG. 2 is a graphical representation of TNF-α-induced apoptosis in C3HA fibroblast cells under various conditions.

FIG. 3 is a graphical representation of lipopolysaccharide-induced PGE₂ production by RAW264.7 macrophages under various conditions.

FIG. 4 is a graphical representation of lipopolysaccharide-induced PGF_{2α} production by RAW264.7 macrophages under various conditions.

FIG. 5 is a graphical representation of lipopolysaccharide-induced PGF_{1α} production by RAW264.7 macrophages under various conditions.

FIGS. 6A and 6B are graphical representations of lipopolysaccharide-induced cytokine production by RAW264.7 macrophages under various conditions from an antibody array study.

FIG. 7 includes graphical representations of the effect of EM-1421 on the replication of influenza virus A/WS/33 in MDCK cells, where panels A and B display the same data but with linear and log y-axes, respectively.

FIG. 8 includes graphical representations of the effect of EM-1421 on the replication of influenza virus A/WS/33 in RAW 264.7 macrophages cells, where panels A and B display the same data but with linear and log y-axes, respectively.

FIG. 9 includes graphical representations of the effect of EM-1421 on the replication of influenza virus A/WS/33 in RAW 264.7 macrophages cells that were treated with EM-1421 prior to virus infection, where panels A and B display the same data but with linear and log y-axes, respectively.

FIG. 10 is a graphical representation of production of TNF-α by RAW264.7 murine macrophages upon viral infection and/or treatment with EM-1421 from a low multiplicity of infection (MOI) model system.

FIG. 11 is a graphical representation of a dose response experiment on the production of TNF-α by RAW264.7 murine macrophages from a low multiplicity of infection (MOI) model system.

FIG. 12 is a graphical representation of a time course experiment on the production of TNF-α by RAW264.7 murine macrophages from a low multiplicity of infection (MOI) model system.

FIG. 13 is a graphical representation of production of TNF-α by RAW264.7 murine macrophages upon viral infection and/or treatment with EM-1421 from a high multiplicity of infection (MOI) model system.

FIG. 14 is a graphical representation of a dose response experiment on the production of TNF-α by RAW264.7 murine macrophages from a high multiplicity of infection (MOI) model system.

FIG. 15 is a graphical representation of a time course experiment on the production of TNF-α by RAW264.7 murine macrophages from a high multiplicity of infection (MOI) model system.

FIG. 16 is a graphical representation of viral infection-induced PGE₂ production by RAW264.7 macrophages under various conditions from a low multiplicity of infection (MOI) model system.

FIG. 17 is a graphical representation of viral infection-induced PGE₂ production by RAW264.7 macrophages under various conditions from a high multiplicity of infection (MOI) model system.

FIG. 18 is a graphical representation of viral infection-induced cytokine production by RAW264.7 macrophages under various conditions from an antibody array study.

### DETAILED DESCRIPTION OF THE PREFERRED EMBODIMENTS

The inventors herein have discovered that catecholic butanes are useful for the treatment of influenza viral infections. The catecholic butanes have the general formula (I) or a pharmaceutically acceptable salt thereof:

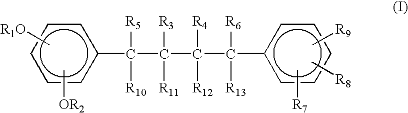

wherein R₁ and R₂ each independently represents a hydrogen, a lower alkyl, a lower acyl, or an alkylene, or —OR₁ and OR₂ each independently represents an unsubstituted or substituted amino acid residue or salt thereof; R₃, R₄, R₅, R₆, R₁₀, R₁₁, R₁₂ and R₁₃ each independently represents a hydrogen, or a lower alkyl; and R₇, R₈ and R₉ each independently represents a hydrogen, —OH, a lower alkoxy, a lower acyloxy, an unsubstituted or substituted amino acid residue or pharmaceutically acceptable salt thereof, or any two adjacent groups together may be an alkyene dioxy; with the proviso that where one of R₇, R₈ and R₉ represents a hydrogen, then —OR₁, —OR₂ and the other two of R₇, R₈ and R₉ do not simultaneously represent —OH. Substituted or unsubstituted amino acid residues and pharmaceutically acceptable salts thereof are preferably bonded to the aromatic ring at their carboxy terminus. Such catecholic butanes can be combined with pharmaceutically acceptable carriers or excipients to produce pharmaceutical compositions that can be formulated for a wide variety of routes of delivery.

In another embodiment of the invention, the catecholic butane has the general formula (I), where R₁ and R₂ are independently —H, a lower alkyl, a lower acyl, or an —OR₁ and —OR₂ each independently represents an unsubstituted or substituted amino acid residue or pharmaceutically acceptable salt thereof; R₃, R₄, are independently a lower alkyl; R₅, R₆, R₁₀, R₁₁, R₁₂ and R₁₃ are independently —H; and R₇, R₈ and R₉ are independently —H, —OH, a lower alkoxy, a lower acyloxy, or unsubstituted or substituted amino acid residue or pharmaceutically acceptable salt thereof; provided that the catecholic butane is not NDGA.

In a further embodiment of the invention, the catecholic butane has the formula (I), where R₁ and R₂ are independently —H, a lower alkyl, a lower acyl, or —OR₁ and —OR₂ each independently represents an unsubstituted or substituted amino acid residue or pharmaceutically acceptable salt thereof; R₃, R₄, are independently a lower alkyl; R₅, R₆, R₇, R₁₀, R₁₁, R₁₂ and R₁₃ are independently —H; and R₈ and R₉ are independently —OH, a lower alkoxy, lower acyloxy, or an unsubstituted or substituted amino acid residue or pharmaceutically acceptable salt thereof; provided that the catecholic butane is not NDGA.

In a further embodiment of the invention, the catecholic butane has the formula (I), where R₁ and R₂ are independently —CH₃ or —(C═O)CH₂N(CH₃)₂ or a salt thereof.

In another embodiment of the invention, the catecholic butane has the formula (I), where R₈ and R₉ are independently —OCH₃ or —O(C═O)CH₂N(CH₃)₂ or a salt thereof.

In a further embodiment of the invention, the catecholic butane has the formula (I), where R₁ and R₂ are independently —CH₃, —(C═O)CH₂N(CH₃)₂ or —(C═O)CH₂N⁺H(CH₃)₂.Cl⁻ and R₈ and R₉ are independently —OCH₃, —O(C═O)CH₂N(CH₃)₂ or —O(C═O)CH₂N⁺H(CH₃)₂.Cl⁻.

In yet another embodiment of the invention, catecholic butane has the formula (I), where R₁ and R₂ are independently —H or —CH₃ and R₈ and R₉ are independently —OH or —OCH₃, provided that the catecholic butane is not NDGA.

In a different embodiment of the invention, the catecholic butane has the formula (I), where R₁ and R₂ are each —CH₃ and R₈ and R₉ are each —OCH₃.

In an alternative embodiment, the catecholic butane used in methods according to embodiments of the present invention is a NDGA derivative with the following formula (II) or a pharmaceutically acceptable salt thereof:

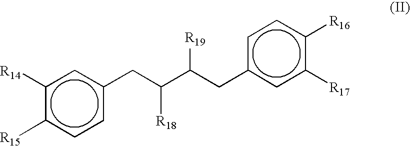

wherein R₁₄, R₁₅, R₁₆ and R₁₇ each independently represents —OH, —OCH₃, —O(C═O)CH₃, or an unsubstituted or substituted amino acid residue or pharmaceutically acceptable salt thereof, R₁₈ and R₁₉ each independently represents —H or an alkyl such as a lower alkyl, for example, —CH₃ or —CH₂CH₃; with the proviso that R₁₄, R₁₅, R₁₆ and R₁₇ are not simultaneously —OH. Substituted or unsubstituted amino acid residues and pharmaceutically acceptable salts thereof are preferably bonded to the aromatic ring at their carboxy terminus.

The present inventors have surprisingly discovered that a composition containing a substantially pure preparation of at least one NDGA derivative is effective for the treatment of influenza viral infections. This finding was serendipitous and surprising as the NDGA derivatives were originally administered for other purposes and influenza treatment was an unexpected realization.

The NDGA derivatives used in embodiments of the present invention preferably have a formula (II) as set forth above, where R₁₄, R₁₅, R₁₆ and R₁₇ each independently represents —OH, a lower alkoxy, for example, —OCH₃, a lower acyloxy, for example, —O(C═O)CH₃, or an unsubstituted or substituted amino acid residue, or pharmaceutically acceptable salt thereof but are not each —OH simultaneously; and R₁₈ and R₁₉ independently represent —H or an alkyl such as a lower alkyl, for example, —CH₃ or —CH₂CH₃. In one embodiment, R₁₈ and R₁₉ can both be —H, —CH₃ or —CH₂CH₃. Preferably, where one or more of R₁₄, R₁₅, R₁₆ and R₁₇ represents an unsubstituted or substituted amino acid residue or salt thereof, the residue is bonded to the aromatic ring at the carboxy terminus.

The present catecholic butane in a suitable formulation, with a pharmaceutically acceptable carrier or excipient where appropriate, can be safely administered to one or more subjects in need of such treatment by one or more routes of administration selected from the group consisting of intranasal administration; oral administration; inhalation administration; subcutaneous administration; transdermal administration; intravenous administration; buccal administration; intraperitoneal administration; intraocular administration; peri-ocular administration; intramuscular administration; implantation administration; infusion, and central venous administration.

Moreover, the catecholic butanes can be safely administered to one or more subjects in need of such treatment in solution, suspension, semisolid or solid forms as appropriate, or in liposomal formulations, nanoparticle formulations, or micellar formulations for administration via one or more routes mentioned above.

Furthermore, the catecholic butanes in liposomal formulations, nanoparticles formulations, or micellar formulations can be embedded in a biodegradable polymer formulation and safely administered, such as by subcutaneous implantation.

In one embodiment of the invention, the route of administration for purposes herein is other than by parenteral administration, where parenteral administration herein means intravenous, intramuscular, subcutaneous, transdermal and intraperitoneal administration.

The present invention further features a pharmaceutical composition containing a catecholic butane for treatment of influenza where the composition is formulated for delivery or administration as described above such as, for example, in the form of a tablet, a capsule, a liquid that is either hydrophilic or hydrophobic, a powder such as one resulting from lyophilization, an aerosol, or in the form of an aqueous water soluble composition, a hydrophobic composition, a liposomal composition, a micellar composition, such as that based on polysorbate 80 or diblock copolymers, a nanoparticle composition, a polymer composition, a cyclodextrin complex composition, emulsions, or lipid based nanoparticles termed “lipocores.”

The present invention additionally provides a pharmaceutical composition containing a catecholic butane for treatment of influenza where the composition is formulated for oral or injectable delivery with a pharmaceutically acceptable carrier, wherein the carrier comprises at least one of a solubilizing agent and an excipient selected from the group consisting of: (a) a water-soluble organic solvent; (b) a cyclodextrin (including a modified cyclodextrin); (c) an ionic, non-ionic or amphipathic surfactant, (d) a modified cellulose; (e) a water-insoluble lipid; and a combination of any of the carriers (a)-(e).

According to embodiments of the present invention, a catecholic butane can be given in combination with one or more other agents or drugs. It can be administered simultaneously, prior to, or following the administration of the other agent or drug. In particular embodiments, a catecholic butane can be administered in combination with one or more additional anti-inflammation agents. The additional anti-inflammation agents are selected from the group consisting of: (1) serotonin receptor antagonists; (2) serotonin receptor agonists; (3) histamine receptor antagonists; (4) bradykinin receptor antagonists; (5) kallikrein inhibitors; (6) tachykinin receptor antagonists, including neurokinin₁ and neurokinin₂ receptor subtype antagonists; (7) calcitonin gene-related peptide (CGRP) receptor antagonists; (8) interleukin receptor antagonists; (9) inhibitors of enzymes active in the synthetic pathway for arachidonic acid metabolites, including (a) phospholipase inhibitors, including PLA₂ isoform inhibitors and PLC_{γ} isoform inhibitors (b) cyclooxygenase inhibitors, and (c) lipooxygenase inhibitors; (10) prostanoid receptor antagonists including eicosanoid EP-1 and EP-4 receptor subtype antagonists and thromboxane receptor subtype antagonists; (11) leukotriene receptor antagonists including leukotriene B₄ receptor subtype antagonists and leukotriene D₄ receptor subtype antagonists; (12) opioid receptor agonists, including mu-opioid, delta-opioid, and kappa-opioid receptor subtype agonists; (13) purinoceptor agonists and antagonists including P_{2X} receptor antagonists and P_{2Y} receptor agonists; (14) adenosine triphosphate (ATP)-sensitive potassium channel openers.

In other embodiments, a catecholic butane can be administered in combination with one or more other anti-influenza agents, such as a second catecholic butane of the general formula I or a pharmaceutically acceptable salt thereof, Amantadine, Oseltamivir, Peramivir, Rimantadine, Zanamivir, or Arbidol.

The present invention further features a method of producing the pharmaceutical composition of the present invention, the method involving making or providing the catecholic butanes in a substantially purified form, combining the composition with a pharmaceutically acceptable carrier or excipient, and formulating the composition in a manner that is compatible with the mode of desired administration.

The present invention still additionally relates to kits comprising compositions or formulations as above for the treatment of influenza where the compositions are formulated for delivery as above, including but not limited to intranasal administration, inhalation, oral administration, topical administration, intravenous administration, intraperitoneal administration and other parenteral administration, optionally, including delivery device for such administration, and instructions for such administration.

### DEFINITIONS

Unless defined otherwise herein, all technical and scientific terms used herein have the same meaning as commonly understood by one of ordinary skill in the art to which this invention belongs. The present invention may be better understood in light of the particular meanings as follows.

The term “active agent,” “compound,” and “drug” as used herein refers to one or more catecholic butanes, including NDGA derivatives, and the pharmaceutically acceptable salt thereof.

The term “alkylene dioxy” as used herein refers to methylene (or substituted methylene) dioxy or ethylene (or substituted ethylene) dioxy.

The “buffer” suitable for use herein includes any buffer conventional in the art, such as, for example, Tris, phosphate, imidazole, and bicarbonate.

A “carrier” as used herein refers to a non-toxic solid, semisolid or liquid filler, diluent, vehicle, excipient, solubilizing agent, encapsulating material or formulation auxiliary of any conventional type, and encompasses all of the components of the composition other than the active pharmaceutical ingredient. The carrier may contain additional agents such as wetting or emulsifying agents, or pH buffering agents. Other materials such as anti-oxidants, humectants, viscosity stabilizers, and similar agents may be added as necessary.

A “cyclodextrin” as used herein means an unmodified cyclodextrin or a modified cyclodextrin, and includes without limitation α-cyclodextrin, β-cyclodextrin, γ-cyclodextrin and any modified cyclodextrins containing modifications thereto, such as hydroxypropyl-β-cyclodextrin (“HP-β-CD”) or sulfobutyl ether β-cyclodextrin (“SBE-β-CD”). Cyclodextrin typically has 6 (α-cyclodextrin), 7 (β-cyclodextrin), and 8 (γ-cyclodextrin) sugars, up to three substitutions per sugar, and 0 to 24 primary substitutions are therefore possible (primary substitutions are defined as substitutions connected directly to the cyclodextrin ring). The modified or unmodified cyclodextrins used in the present invention may have any appropriate number and location of primary substitutions or other modifications.

The term “cytokine” as used herein means any of numerous hormone-like, low-molecular-weight proteins, secreted by various cell types, which regulate the intensity and duration of immune response and mediate cell-to-cell communication during immunoregulatory and inflammatory processes. Examples of cytokines include chemokines, interleukins, lymphokines, other signaling molecules such as tumor necrosis factor and interferons, etc.

The term “chemokine” as used herein means a group of small, mostly basic, structurally related molecules that regulate cell trafficking of various types of leukocytes through interactions with a subset of 7-transmembrane, G protein-coupled receptors. Chemokines also play fundamental roles in the development, homeostasis, and function of the immune system, and they have effects on cells of the central nervous system as well as on endothelial cells involved in angiogenesis or angiostasis.

The term “interleukin” or “IL” as used herein means a group of multifunctional cytokines that are synthesized by lymphocytes, monocytes, macrophages, and certain other cells.

The term “lymphokine” as used herein means a group of cytokines released by activated lymphocytes, which mediates immune response.

The term “interferon” as used herein means a group of glycoprotein secreted by vertebrate cells in response to a wide variety of challenges by foreign agents such as viruses, bacteria, parasites and tumor cells. Interferons assist the immune response and confer resistance against the foreign agents, for example, by inhibiting proliferation of normal and malignant cells, impeding multiplication of intracellular parasites, enhancing macrophage and granulocyte phagocytosis, augmenting natural killer cell activity, and having several other immunomodulatory functions.

The term “tumor necrosis factor” or “TNF” as used herein means a cytokine mainly secreted by macrophages. A TNF can bind to, and thus functions through its receptors TNFRSF1A/TNFR1 and TNFRSF1B/TNFBR. This cytokine is involved in the regulation of a wide spectrum of biological processes including cell proliferation, differentiation, apoptosis, lipid metabolism, and coagulation. This cytokine has been implicated in a variety of diseases, including autoimmune diseases, insulin resistance, and cancer. The increased production of TNF upon influenza viral infection has also been implicated in the manifestation of diseases, disorders, or syndromes associated with the viral infection (see descriptions infra).

The term “unsubstituted or substituted amino acid residue or salt thereof” as used herein in reference to one of the —OR₁, —OR₂ or other R groups as appropriate, in the formulas for the catecholic butanes herein is an amino acid residue or a substituted amino acid residue or salt of an amino acid residue or salt of a substituted amino acid residue including but not limited to: alanine, arginine, asparagine, aspartate, cysteine, glutamate, glutamine, glycine, histidine, isoleucine, leucine, lysine, methionine, phenylalanine, proline, serine, threonine, tryptophan, tyrosine, valine, 5-hydroxylysine, 4-hydroxyproline, thyroxine, 3-methylhistidine, ε-N-methyllysine, ε-N,N,N-trimethyllysine, aminoadipic acid, γ-caroxyglutamic acid, phosphoserine, phosphothreonine, phosphotyrosine, N-methylarginine, N-acetyllysine, and an N,N-dimethyl-substituted amino acid residue, or a pharmaceutically acceptable salt thereof.

The term “lower alkyl” as used herein means a C₁-C₆ alkyl, which may be linear or branched and which may optionally include one or more unsaturated carbon-carbon bonds.

The term “lower acyl” as used herein means a C₁-C₆ acyl, which may be linear or branched and which may optionally include one or more unsaturated carbon-carbon bonds.

The term “NDGA” as used herein refers to nordihydroguaiaretic acid.

The term “NDGA derivative” as used herein refers to one or more compounds each having the formula (II), or a pharmaceutically acceptable salt thereof:

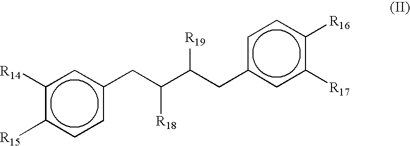

wherein R₁₄, R₁₅, R₁₆ and R₁₇ are independently —OH, lower alkoxy, lower acyloxy, or an unsubstituted or substituted amino acid residue, or pharmaceutically acceptable salt thereof, but are not each —OH simultaneously; and R₁₈ and R₁₉ are independently —H or an alkyl such as a lower alkyl. The term includes, for example, a compound in which R₁₄, R₁₅, R₁₆ and R₁₇ are each —OCH₃, or are each —O(C═O)CH₃; and R₁₈ and R₁₉ are each —H or each a lower alkyl. In one embodiment of the invention, R₁₈ and R₁₉ are each —CH₃ or —CH₂CH₃.

A “pharmaceutically acceptable carrier” as used herein refers to a non-toxic solid, semisolid or liquid filler, diluent, encapsulating material or formulation auxiliary of any conventional type. A “pharmaceutically acceptable carrier” is non-toxic to recipients at the dosages and concentrations employed, and is compatible with other ingredients of the formulation. For example, the carrier for a formulation containing the present catecholic butane preferably does not include oxidizing agents and other compounds that are known to be deleterious to such. Suitable carriers include, but are not limited to, water, dextrose, glycerol, saline, ethanol, buffer, dimethyl sulfoxide, Cremaphor EL, and combinations thereof. The carrier may contain additional agents such as solubilizing, wetting or emulsifying agents, or pH buffering agents. Other materials such as anti-oxidants, humectants, viscosity stabilizers, and similar agents may be added as necessary.

Pharmaceutically acceptable salts as used herein include the acid addition salts (formed with the free amino groups of the polypeptide) and which are formed with inorganic acids such as, for example, hydrochloric or phosphoric acids, or such organic acids as acetic, mandelic, oxalic, and tartaric acids. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, and histidine.

The term “pharmaceutically acceptable excipient” as used herein includes vehicles, adjuvants, or diluents or other auxiliary substances, such as those conventional in the art, which are readily available to the public. For example, pharmaceutically acceptable auxiliary substances include pH adjusting and buffering agents, tonicity adjusting agents, stabilizers, wetting agents and the like.

The terms “subject,” “host,” and “patient,” as used herein are used interchangeably to refer to an animal being treated with the present compositions, including, but not limited to, simians, humans, avians, felines, canines, equines, rodents, bovines, porcines, ovines, caprines, mammalian farm animals, mammalian sport animals, and mammalian pets.

A “substantially purified” as used herein compound in reference to the catecholic butanes is one that is substantially free of compounds that are not the catecholic butane of the present invention (hereafter, “non-NDGA materials”). By substantially free is meant at least 50%, preferably at least 70%, more preferably at least 80%, and even more preferably at least 90% free of non-NDGA materials.

As used herein, the terms “treatment,” “treating,” and the like, as used herein refer to obtaining a desired pharmacologic and/or physiologic effect. The effect may be prophylactic in terms of completely or partially preventing a condition or disease or symptom thereof and/or may be therapeutic in terms of a partial or complete cure for a condition or disease and/or adverse affect attributable to the condition or disease. “Treatment,” thus, for example, covers any treatment of a condition or disease in a mammal, particularly in a human, and includes: (a) preventing the condition or disease or symptom thereof from occurring in a subject which may be predisposed to the condition or disease but has not yet been diagnosed as having it; (b) inhibiting the condition or disease or symptom thereof, such as, arresting its development; and (c) relieving, alleviating or ameliorating the condition or disease or symptom thereof, such as, for example, causing regression of the condition or disease or symptom thereof.

The term “therapeutically effective amount” or “effective amount” as used herein, means that amount of an active agent, a compound, or a drug, that elicits a desired biological or medicinal response in a tissue system of a subject, or in a subject, that is being sought by a researcher, veterinarian, medical doctor or other clinician. The desired response includes interdicting, preventing, palliating, or alleviating an existing viral infection in the subject that is being treated. In some embodiments, the desired response includes at least a reduction in one or more symptoms, disorders, or diseases of influenza viral infection in the subject under treatment. In some other embodiments, the desired response includes a reduction in the count of virus, or a inhibition of the replication or growth of an influenza virus in the subject under treatment.

One skilled in the art will recognize that the “therapeutically effective amount” of an active agent to be used in the instant invention can vary with factors, such as the particular subject, e.g., age, weight, diet, health, etc., severity and complications of viral infection condition sought to be treated or prevented, the mode of administration of the active agent, the particular active agent used, etc. Standard procedures can be performed to evaluate the effect of the administration of an active agent to a subject, thus allowing a skilled artisan to determine the effective amount of the active agent to be administered to the subject. For example, the syndrome of viral infection such as fever or inflammation, etc., or the count of virus, can be measured from the subject prior to or after the administration of the active agent. In addition, techniques, such as surveys or animal models, can also be used to evaluate the effectiveness of an active agent in treating or preventing a viral infection.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All publications mentioned herein, including patents, patent applications, and journal articles are incorporated herein by reference in their entireties including the references cited therein, which are also incorporated herein by reference. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

It must be noted that as used herein, the singular forms “a”, “an”, and “the” include plural referents unless the context clearly dictates otherwise. Thus, for example, reference to “a compound” includes a plurality of such compounds and reference to “the catecholic butane” includes reference to one or more catecholic butanes and equivalents thereof known to those skilled in the art.

The embodiments of the invention described below are given by way of example only and is not to be interpreted in any way as limiting the invention.

Preparation of Catecholic Butanes:

The catecholic butanes of the present invention can be prepared by any conventional methodologies. For example, such compounds can be made as described in U.S. Pat. No. 5,008,294 (Jordan et al., issued Apr. 16, 1991); U.S. Pat. No. 6,291,524 (Huang et al., issued Sep. 18, 2001); Hwu, et al. (Hwu, J. R. et al., “Antiviral activities of methylated nordihydroguaiaretic acids. 1. Synthesis, structure identification, and inhibition of Tat-regulated HIV transactivation. J. Med. Chem., 41(16): 2994-3000” (1998)); or McDonald, et al. (McDonald, R. W. et al., ““Synthesis and anticancer activity of nordihydroguaiaretic acid (NDGA) and analogues.” Anti-Cancer Drug Des., 16: 261-270 (2001)).

In one embodiment of the present invention, a catecholic butane, tetra-O-methyl NDGA, also known as meso-1,4-bis(3,4-dimethoxyphenyl)-2,3-dimethylbutane, terameprocol, EM-1421 or M₄N (as shown in the formula below), was made as follows: a solution was made containing NDGA and potassium hydroxide in methanol in a reaction flask. Dimethyl sulfate was then added to the reaction flask and the reaction was allowed to proceed. The reaction was finally quenched with water, causing the product to precipitate. The precipitate was isolated by filtration and dried in a vacuum oven. The compound was then dissolved in a solution of methylene chloride and toluene and subsequently purified through an alumina column. The solvents were removed by rotary evaporation and the solid was resuspended in isopropanol and isolated by filtration. The filter cake was dried in a vacuum oven. The resulting tetra-O-methyl NDGA (M₄N) was crystallized by refluxing the filter cake in isopropanol and re-isolating the crystals by filtration.

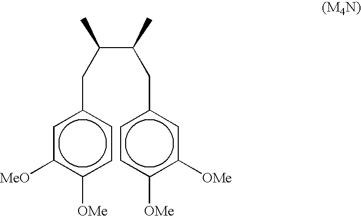

In some embodiments of the present invention, certain catecholic butanes of the present invention, such as G₄N, also known as meso-1,4-bis[3,4-(dimethylaminoacetoxy)phenyl]-(2R,3S)-dimethylbutane or tetra-dimethylglycinyl NDGA (shown in the formula below), or a hydrochloride salt thereof and similar compounds having amino acid substituents, can also be prepared according to conventional methods, as described in, for example, U.S. Pat. No. 6,417,234.

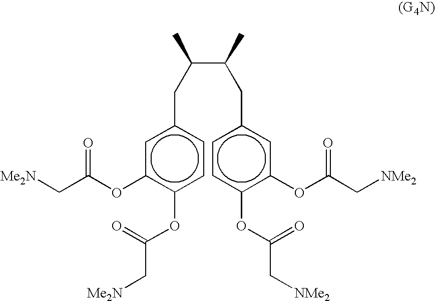

Compositions:

The present invention further provides compositions, including pharmaceutical compositions, comprising the catecholic butanes and pharmaceutically acceptable carriers or excipients. These compositions may include a buffer, which is selected according to the desired use of the catecholic butanes, and may also include other substances appropriate for the intended use. Those skilled in the art can readily select an appropriate buffer, a wide variety of which are known in the art, suitable for an intended use. In some instances, the composition can comprise a pharmaceutically acceptable excipient, a variety of which are known in the art. Pharmaceutically acceptable excipients suitable for use herein are described in a variety of publications, including, for example, Gennaro (Gennaro, A., “Remington: The Science and Practice of Pharmacy”, 19th edition, Lippincott, Williams, & Wilkins, (1995)); Ansel, et al. (Ansel, H. C. et al., “Pharmaceutical Dosage Forms and Drug Delivery Systems eds., 7^{th} ed., Lippincott, Williams, & Wilkins (1999)); and Kibbe (Kibbe, A. H., Handbook of Pharmaceutical Excipients, 3^{rd} ed. Amer. Pharmaceutical Assoc.).

The compositions herein are formulated in accordance to the mode of potential administration. Thus, if the composition is intended to be administered intranasally or by inhalation, for example, the composition may be a converted to a powder or aerosol form, as conventional in the art, for such purposes. Other formulations, such as for oral or parenteral delivery, are also used as conventional in the art.

Compositions for administration herein may form solutions, suspensions, tablets, pills, capsules, sustained release formulations or powders.

Compositions or formulations suitable for oral or injectable delivery additionally includes a pharmaceutical composition containing a catecholic butane for treatment of influenza where the composition is formulated with a pharmaceutically acceptable carrier, wherein the carrier comprises at least one of a solubilizing agent and an excipient selected from the group consisting of: (a) a water-soluble organic solvent; (b) a cyclodextrin (including a modified cyclodextrin); (c) an ionic, non-ionic or amphipathic surfactant, (d) a modified cellulose; (e) a water-insoluble lipid; and a combination of any of the carriers (a)-(e).

The water-soluble organic solvent may be preferably, but not necessarily, other than dimethyl sulfoxide. Non-limiting exemplary water-soluble organic insolvents include polyethylene glycol (“PEG”), for example, PEG 300, PEG 400 or PEG 400 monolaurate, propylene glycol (“PG”), polyvinyl pyrrolidone (“PVP”), ethanol, benzyl alcohol or dimethylacetamide. Preferably, for certain embodiments, when the water-soluble organic solvent is PG, the PG is in the absence of white petrolatum, in the absence of xanthan gum (also known as xantham gum and xanthum gum) and in the absence of at least one of glycerine or glycine. When the water-soluble organic solvent is PEG, for certain embodiments, it is preferred that the PEG is present in the absence of ascorbic acid or butylated hydroxytoluene (“BHT”), and for certain embodiments, when the PEG is polyethylene glycol 400, the polyethylene glycol 400 preferably is present in the absence of polyethylene glycol 8000.

The cyclodextrin or modified cyclodextrin may be, without limitation, α-cyclodextrin, β-cyclodextrin, γ-cyclodextrin, HP-β-CD or SBE-β-CD.

The ionic, non-ionic or amphipathic surfactant may include, for example without limitation, a surfactant such as polyoxyethylene sorbitan monolaurate (also known as polysorbate), which is a non-ionic surfactant, for example, polysorbate 20 and polysorbate 80, commercially available as Tween® 20 or Tween® 80, d-alpha-tocopheryl polyethylene glycol 1000 succinate (“TPGS”), glycerol monooleate (also known as glyceryl monooleate), an esterified fatty acid or a reaction product between ethylene oxide and castor oil in a molar ratio of 35:1, commercially available as Cremophor® EL. Preferably, for certain embodiments, when the surfactant is a non-ionic surfactant, the non-ionic surfactant is present in the absence of xanthan gum.

Non-limiting examples of a modified cellulose, include ethyl cellulose (“EC”), hydroxylpropyl methylcellulose (“HPMC”), methylcellulose (“MC”) or carboxy methylcellulose (“CMC”). In one embodiment of the invention, the catecholic butane may be solubilized in modified celluloses that can be diluted in ethanol (“EtOH”) prior to use.

The water-insoluble lipids include, for example, an oil or oils, such as castor oil, sesame oil or peppermint oil, a wax or waxes, such as beeswax or carnuba wax, and mixed fat emulsion compositions such as Intralipid® (Pharmacia & Upjohn, now Pfizer), used as per the manufacturer's recommendation. For example, adult dosage is recommended to be not exceeding 2 g of fat/kg body weight/day (20 mL, 10 mL and 6.7 mL/kg of Intralipid® 10%, 20% and 30%, respectively). Intralipid® 10% is believed to contain in 1,000 mL: purified soybean oil 100 g, purified egg phospholipids 12 g, glycerol anhydrous 22 g, water for injection q.s. ad 1,000 mL. pH is adjusted with sodium hydroxide to pH approximately 8. Intralipid® 20% contains in 1,000 mL: purified soybean oil 200 g, purified egg phospholipids 12 g, glycerol anhydrous 22 g, water for injection q.s. ad 1,000 mL. pH is adjusted with sodium hydroxide to pH approximately 8. Intralipid® 30% contains in 1,000 mL: purified soybean oil 300 g, purified egg phospholipids 12 g, glycerol anhydrous 16.7 g, water for injection q.s. ad 1,000 mL. pH is adjusted with sodium hydroxide to pH approximately 7.5. These Intralipid® products are stored at controlled room temperature below 25° C. and should not be frozen. For certain embodiments of injectable formulations, the oil is an oil other than castor oil, and for certain embodiments of oral formulations, the castor oil is present in the absence of beeswax or carnuba wax.

In one embodiment of the invention, the catecholic butane is dissolved or dissolved and diluted in different carriers to form a liquid composition for oral administration into animals, including humans. For example, in one aspect of this embodiment, the catecholic butane is dissolved in a water-soluble organic solvent such as a PEG 300, PEG 400 or a PEG 400 monolaurate (the “PEG compounds”) or in PG. In another embodiment, the compounds herein are dissolved in a modified cyclodextrin, such as HP-β-CD or SBE-β-CD. In yet another embodiment, the present compounds are solubilized and/or diluted in a combination formulation containing a PEG compound and HP-β-CD. In a further embodiment, the compounds herein are dissolved in a modified cellulose such as HPMC, CMC or EC. In yet another embodiment, the compounds herein are dissolved in another combination formulation containing both a modified cyclodextrin and modified cellulose, such as, for example, HP-β-CD and HPMC or HP-β-CD and CMC.

In yet another embodiment, the compounds herein are dissolved in ionic, non-ionic or amphipathic surfactants such as Tween® 20, Tween® 80, TPGS or an esterified fatty acid. For example, the present compounds can be dissolved in TPGS alone, or Tween® 20 alone, or in combinations such as TPGS and PEG 400, or Tween® 20 and PEG 400.

In a further embodiment, the present compounds are dissolved in a water-insoluble lipid such as a wax, fat emulsion, for example Intralipid®, or oil. For example, the present compounds can be dissolved in peppermint oil alone, or in combinations of peppermint oil with Tween® 20 and PEG 400, or peppermint oil with PEG 400, or peppermint oil with Tween® 20, or peppermint oil with sesame oil.

Of course, EC may be substituted or added in place of the HPMC or CMC in the foregoing examples; PEG 300 or PEG 400 monolaurate can be substituted or added in place of PEG 400 in the foregoing examples; Tween® 80 may be substituted or added in place of Tween® 20 in the foregoing examples; and other oils such as corn oil, olive oil, soybean oil, mineral oil or glycerol, may be substituted or added in place of the peppermint oil or sesame oil in the foregoing examples.

Further, heating may be applied, for example, heating to a temperature of about 30° C. to about 90° C., in the course of formulating any of these compositions to achieve dissolution of the compounds herein or to obtain an evenly distributed suspension of the present compounds.

In still a further embodiment, the catecholic butane may be administered orally as solids either without any accompanying carrier or with the use of carriers. In one embodiment, the compounds herein are first dissolved in a liquid carrier as in the foregoing examples, and subsequently made into a solid composition for administration as an oral composition. For example, the present compounds are dissolved in a modified cyclodextrin such as HP-β-CD, and the composition is lyophilized to yield a powder that is suitable for oral administration.

In a further embodiment, the present compounds are dissolved or suspended in a TPGS solution, with heating as appropriate to obtain an evenly distributed solution or suspension.

Upon cooling, the composition becomes creamy and is suitable for oral administration. In yet another embodiment, the present compounds are dissolved in oil and beeswax is added to produce a waxy solid composition.

In general, in preparing the oral formulations, the compounds herein are first solubilized before other excipients are added so as to produce compositions of higher stability. Unstable formulations are not desirable. Unstable liquid formulations frequently form crystalline precipitates or biphasic solutions. Unstable solid formulations frequently appear grainy and clumpy and sometimes contain runny liquids. An optimal solid formulation appears smooth, homogenous, and has a small melting temperature range. In general, the proportions of excipients in the formulation may influence stability. For example, too little stiffening agent such as beeswax may leave the formulation too runny for an elegant oral formulation.

Hence, in general, for the liquid formulations of the present invention, the excipients used should be good solvents of the catecholic butane compounds herein, such as M₄N, for example. In other words, the excipients should be able to dissolve the catecholic butane without heating. The excipients should also be compatible with each other independent of the catecholic butane such that they can form a stable solution, suspension or emulsion. Also, in general, for the solid formulations of the present invention, the excipients used should also be good solvents of the catecholic butane to avoid clumps and non-uniform formulations. To avoid solid formulations that are too runny or heterogeneous in texture, which are undesirable, the excipients should be compatible with each other such that they form a smooth homogeneous solid, even in the absence of the catecholic butane.

Therapeutic Methods:

The catecholic butanes and compositions of the subject invention find use as therapeutic agents in situations where one wishes to provide a treatment to a subject suffering from an influenza viral infection.

A variety of animal hosts are treatable according to the subject methods, including human and non-human animals, such as birds in the case of avian influenza, where there is concern about trans-species infection from birds to mammals in general and humans in particular. Generally such hosts are “mammals” or “mammalian,” where these terms are used broadly to describe organisms which are within the class mammalia, including the orders carnivore (e.g., dogs and cats), rodentia (e.g., guinea pigs, and rats), and other mammals, including cattle, goats, horses, sheep, rabbits, pigs, and primates (e.g., humans, chimpanzees, and monkeys). In many embodiments, the hosts will be humans. Animal models are of interest for experimental investigations, such as providing a model for treatment of human disease. Further, the present invention is applicable to veterinary care as well.

Formulations, Dosages, and Routes of Administration:

As mentioned above, an effective amount of the active agent is administered to the host or the subject. Typically, the compositions of the instant invention will contain from less than about 1% up to about 99% of the active ingredient, that is, the catecholic butanes herein; optionally, the instant invention will contain about 5% to about 90% of the active ingredient. The present invention additionally provides compositions in which the active agents, such as the catecholic butanes, including the NDGA derivatives, for example, M₄N, are administered to subjects, such as humans, at an oral dose of about less than 0.1 mg/kg to about 400 mg/kg or more based on the weight of the animals, such as humans, for example. More specifically and only by way of example without limitation, the subjects may be treated via any suitable route of administration, with a range from about 0.01 to about 400 mg/kg of body weight per dose, such as less than about 0.01 mg/kg, 0.05 mg/kg, 0.1 mg/kg, 0.5 mg/kg, 1.0 mg/kg, 2.5 mg/kg, 5.0 mg/kg, 10 mg/kg, 15 mg/kg, 25 mg/kg, 50 mg/kg, 100 mg/kg, 150 mg/kg, 200 mg/kg, 250 mg/kg, 300 mg/kg, 350 mg/kg, or 400 mg/kg, or more.

The appropriate dose to be administered depends on the subject to be treated, such as the general health of the subject, the age of the subject, the state of the disease or condition, the weight of the subject, for example. Generally, about 0.1 mg to about 500 mg may be administered to a child and about 0.1 mg to about 5 grams may be administered to an adult. The active agent can be administered in a single or, more typically, multiple doses. Preferred dosages for a given agent are readily determinable by those of skill in the art by a variety of means. Other effective dosages can be readily determined by one of ordinary skill in the art through routine trials establishing dose response curves. The amount of agent will, of course, vary depending upon the particular agent used.

The frequency of administration of the active agent, as with the doses, will be determined by the care giver based on age, weight, disease status, health status and patient responsiveness. Thus, the agents may be administered continuously, intermittently, one or more times daily or in other periods as appropriate for as long as needed as conventionally determined.

The catecholic butanes or active agents of the present invention can be incorporated into a variety of formulations for therapeutic administration. More particularly, the catecholic butanes of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, aerosols, liposomes, nanoparticles, granules, ointments, solutions, suppositories, injections, inhalants and aerosols.

As such, administration of the active agents can be achieved in various ways, such as oral, buccal, rectal, intranasal, intravenous, subcutaneous, intramuscular, intra-tracheal, topical, interstitial, transdermal, etc., or by inhalation or implantation. In particular, nanoparticle, micelle and liposomal preparation can be administered systemically, including parenterally and intranasally, as well as interstitially, orally, topically, transdermally, via inhalation or implantation, such as for drug targeting, enhancement of drug bioavailability and protection of drug bioactivity and stability. Nanoparticle bound drugs herein are expected to achieve prolonged drug retention in vivo.

In pharmaceutical dosage forms, the active agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the active agents can be used alone or in combination with appropriate additives as liquids in the form of solutions or suspensions or as solids in the form of tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The pharmaceutically acceptable excipients, such as vehicles, adjuvants, carriers or diluents, are conventional in the art. Suitable excipient vehicles are, for example, water, saline, dextrose, glycerol, ethanol, or the like, and combinations thereof. In addition, if desired, the vehicle may contain minor amounts of auxiliary substances such as pH adjusting and buffering agents, tonicity adjusting agents, stabilizers, wetting agents or emulsifying agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The active agents can be formulated into preparations for injection by dissolving, suspending or emulsifying them in an aqueous or non-aqueous solvent, such as vegetable or other similar oils, including corn oil, castor oil, synthetic aliphatic acid glycerides, esters of higher aliphatic acids or propylene glycol; and if desired, with conventional additives such as solubilizers, isotonic agents, suspending agents, emulsifying agents, stabilizers and preservatives. Suitable therapeutic formulations for parenteral delivery of a catecholic butane in accordance with the present invention also include the various injectable carrier/excipient formulations disclosed in U.S. Provisional Patent Application No. 60/647,648 filed Jan. 27, 2005, and an International Application No. PCT/US2006/00287 filed Jan. 27, 2006, entitled “Formulations for Injection of Catecholic Butanes, Including NDGA Compounds, Into Animals,” International Publication No. WO2006/081364A2 published Aug. 3, 2006, the entire contents of which are incorporated herein by reference.

The active agents can be utilized in aerosol formulation to be administered via inhalation. The compounds of the present invention can be formulated into pressurized acceptable propellants such as dichlorodifluoromethane, propane, nitrogen and the like.

Furthermore, the active agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise the active agent(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Kits with multiple or unit doses of the active agent, are included in the present invention. In such kits, in addition to the containers containing the multiple or unit doses of the compositions containing the NDGA derivatives will be an informational package insert with instructions describing the use and attendant benefits of the drugs in treating the pathological condition of interest, in this case, influenza and particularly influenza subtype H5N1.

Preparation of NanoParticles (“NP”):

The present invention includes formulations of catecholic butanes in a NP preparation. A number of different NP formulations suitable for use herein can be made depending on the method of delivery. The NP formulation can differ based on the drug release profile desired, by controlling the molecular weight, the copolymer ratio, the drug loading, the microparticle size and porosity and the fabrication conditions. The NP formulations can also differ on the basis of polymers, stabilizers, and surfactants used in the production process. Different excipients may also have different effects on drug uptake, drug distribution throughout the body and persistence of the drug in plasma. A person having skills conventional in the art will be able to determine the desired properties or characteristics, and accordingly determine the appropriate NP formulation to use.

The polymeric matrix of the NP must meet the criteria of biocompatibility, bioavailability, mechanical strength and ease of processing. The best known polymers for this purpose is the biodegradable poly(lactide-co-glycolide)s (“PLGAs”).

NP herein can be made by any process conventional in the art. In one embodiment, the NP can be made as described in, for example, Lockman, et al. (Lockman, P. R. et al., “Nanoparticle Technology for Drug Delivery Across the Blood-Brain Barrier.”, Drug Development Indus. Pharmacy, 28(1): 1-13, (2002)). The types of manufacturing process include, for example, emulsion polymerization, interfacial polymerization, desolvation evaporation and solvent deposition.

In the emulsion polymerization process of making the NP herein, the polymerization process consists of building a chain of polymers from a single monomer unit, as described in, for example, Kreuter (Kreuter, J., “Nanoparticles, In Encyclopedia of Pharmaceutical Technology, Swarbick, J.; Boylan, J. C. Eds.; Marcel Dekker (New York, 1994), pp. 165-190, (1994)). Polymerization occurs spontaneously at room temperature after initiation by either free radical or ion formation, such as by use of high-energy radiation, UV light, or hydroxyl ions. Once polymerization is complete the solution is filtered and neutralized. The polymers form micelles and droplets consisting of from about 100 to 10⁷ polymer molecules. Surfactants and stabilizers are generally not need in this process. Also, this process can be accomplished in an organic phase rather than an aqueous phase.

The NP herein can also be made by an interfacial polymerization process as described in, for example, Khouri (Khouri, A. I. et al., “Development of a new process for the manufacture of polyisobutyl-cyanoacrylate nanoparticles,” Int. J. Pharm., 28: 125 (1986)). In this process, monomers are used to create the polymer and polymerization occurs when an aqueous and organic phase are brought together by homogenization, emulsification, or micro-fluidization under high-torque mechanical stirring. For example, polyalkylcyanoacrylate nanocapsules containing the catecholic butanes can be made by combining the lipophilic catecholic butanes and the monomer in an organic phase, dissolving the combination in oil, and slowly adding the mixture through a small tube to an aqueous phase with constant stirring. The monomer can then spontaneously form 200-300 nm capsules by anionic polymerization. A variation of this process involves adding a solvent mixture of benzyl benzoate, acetone, and phospholipids to the organic phase containing the monomer and the drug, as described in, for example, Fessi, et al. (Fessi, H. et al., “Nanocapsule formulation by interfacial deposition following solvent displacement.”, Int. J. Pharm., 55: R1-R4, (1989)). This creates a formulation in which the drug is encapsulated and protected against degradation until it reaches the target tissue.

Macromolecules such as albumin and gelatin can be used in oil denaturation and desolvation processes in the production of NPs. In the oil emulsion denaturation process, large macromolecules are trapped in an organic phase by homogenization. Once trapped, the macromolecule is slowly introduced to an aqueous phase undergoing constant stirring. The nanoparticles formed by the introduction of the two immiscible phases can then be hardened by crosslinking, such as with an aldehyde or by heat denaturation.

Alternatively, macromolecules can form NPs by “desolvation.” In the desolvation process, macromolecules are dissolved in a solvent in which the macromolecules reside in a swollen, coiled configuration. The swollen macromolecule is then induced to coil tightly by changing the environment, such as pH, charge, or by use of a desolvating agent such as ethanol. The macromolecule may then be fixed and hardened by crosslinking to an aldehyde. The NDGA Compounds can be adsorbed or bound to the macromolecule before crosslinking such that the derivatives become entrapped in the newly formed particle.

Solid lipid NP can be created by high-pressure homogenization. Solid lipid NPs have the advantage that they can be sterilized and autoclaved and possess a solid matrix that provides a controlled release.

The present invention further includes NP with different methods of drug loading. The NP can be solid colloidal NP with homogeneous dispersion of the drug therein. The NP can be solid NP with the drug associated on the exterior of the NP, such as by adsorption. The NP can be a nanocapsule with the drug entrapped therein. The NP can further be solid colloidal NP with homogeneous dispersion of the drug therein together with a cell surface ligand for targeting delivery to the appropriate tissue.

The size of the NPs may be relevant to their effectiveness for a given mode of delivery. The NPs typically are about 10 nm to about 1000 nm; optionally, the NPs can be about 30 nm to about 800 nm; further typically, about 60 nm to about 270 nm; even further typically, about 80 nm to about 260 nm; or about 90 nm to about 230 nm, or about 100 nm to about 195 nm. Several factors influence the size of the NPs, all of which can be adjusted by a person of ordinary skill in the art, such as, for example, pH of the solution used during polymerization, amount of initiation triggers (such as heat or radiation, etc.) and the concentration of the monomer unit. Sizing of the NPs can be performed by photon correlation spectroscopy using light scattering.

The NPs herein, such as polysaccharide NPs or albumin NPs, may optionally be coated with a lipid coating. For example, polysaccharide NPs can be crosslinked with phosphate (anionic) and quarternary ammonium (cationic) ligands, with or without a lipid bilayer, such as one containing dipalmitoyl phosphatidyl choline and cholesterol coating. Other polymer/stabilizer include, but is not limited to: soybean oil; maltodextrin; polybutylcyanoacrylate; butylcayanoacrylate/dextran 70 kDa, Polysorbate-85; polybutylcyanoacrylate/dextran 70 kDa, Polysorbate-85; stearic acid; poly-methylmethylacrylate.

The NP preparations containing the catecholic butanes such as by adsorption to the NPs, can be administered intravenously for treatment of influenza. To avoid undesirable uptake of these NP preparations by the reticuloendothelial cells, the NPs may be coated with a surfactant or manufactured with a magnetically responsive material.

Thus, optionally, a surfactant may be used in conjunction with the NP. For example, polybutylcyanoacrylate NPs can be used with a dextran-70,000 stabilizer and Polysorbate-80 as a surfactant. Other surfactants include, but not limited to: Polysorbate-20, 40, or 60; Poloxamer 188; lipid coating-dipalmitoyl phosphatidylcholine; Epikuron 200; Poloxamer 338; Polaxamine 908; Polaxamer 407. For example, Polyaxamine 908 may be used as a surfactant to decrease uptake of NPs into the RES of the liver, spleen, lungs, and bone marrow.

The magnetically responsive material can be magnetite (Fe₃O₄) which can be incorporated into the composition for making the NP. These magnetically responsive NPs can be externally guided by a magnet.

In another embodiment, the NPs herein can be made as described in Mu and Feng using a blend of poly(lactide-co-glycolide)s (“PLGAs”) and d-α-tocopheryl polyethylene glycol 1000 succinate (vitamin E TPGS or TPGS) (Mu, L. and Feng, S. S., “A novel controlled release formulation for the anticancer drug paclitaxel (Taxol®): PLGA nanoparticles containing vitamin E TPGS.” J. Control. Rel. 86: 33-48 (2003)). The latter can also act as an emulsifier, in addition to being a matrix material.

Preparation of Micelle Forming Carriers:

The present invention includes catecholic butanes formulated in micelle forming carriers, where the micelles are produced by processes conventional in the art. Examples of such are described in, for example, Liggins (Liggins, R. T. and Burt, H. M., “Polyether-polyester diblock copolymers for the preparation of paclitaxel loaded polymeric micelle formulations.” Adv. Drug Del. Rev. 54: 191-202, (2002)); Zhang, et al. (Zhang, X. et al., “Development of amphiphilic dibiock copolymers as micellar carriers of taxol.” Int. J. Pharm. 132: 195-206, (1996)); and Churchill (Churchill, J. R., and Hutchinson, F. G., “Biodegradable amphipathic copolymers.” U.S. Pat. No. 4,745,160, (1988)). In one such method, polyether-polyester block copolymers, which are amphipathic polymers having hydrophilic (polyether) and hydrophobic (polyester) segments, are used as micelle forming carriers.

Another type of micelles is, for example, that formed by the AB-type block copolymers having both hydrophilic and hydrophobic segments, which are known to form micellar structures in aqueous media due to their amphiphilic character, as described in, for example, Tuzar (Tuzar, Z. and Kratochvil, P., “Block and graft copolymer micelles in solution.”, Adv. Colloid Interface Sci. 6:201-232, (1976)); and Wilhelm, et al. (Wilhelm, M. et al., “Poly(styrene-ethylene oxide) block copolymer micelle formation in water: a fluorescence probe study.”, Macromolecules 24: 1033-1040 (1991)). These polymeric micelles are able to maintain satisfactory aqueous stability irrespective of the high content of hydrophobic drug incorporated within the micelle inner core. These micelles, in the range of approximately <200 nm in size, are effective in reducing non-selective RES scavenging and show enhanced permeability and retention.

Further, for example, poly(D,L-lactide)-b-methoxypolyethylene glycol (MePEG:PDLLA) diblock copolymers can be made using MePEG 1900 and 5000. The reaction can be allowed to proceed for 3 hr at 160° C., using stannous octoate (0.25%) as a catalyst. However, a temperature as low as 130° C. can be used if the reaction is allowed to proceed for about 6 hr, or a temperature as high as 190° C. can be used if the reaction is carried out for only about 2 hr.

In one embodiment, N-isopropylacrylamide (“IPAAm”) (Kohjin, Tokyo, Japan) and dimethylacrylamide (“DMAAm”) (Wako Pure Chemicals, Tokyo, Japan) can be used to make hydroxyl-terminated poly(IPAAm-co-DMAAm) in a radical polymerization process, using the method of Kohori, F. et al. (1998). (Kohori, F. et al., “Preparation and characterization of thermally Responsive block copolymer micelles comprising poly(N-isopropylacrylamide-b-D,L-lactide).” J. Control. Rel. 55: 87-98, (1998)). The obtained copolymer can be dissolved in cold water and filtered through two ultrafiltration membranes with a 10,000 and 20,000 molecular weight cut-off. The polymer solution is first filtered through a 20,000 molecular weight cut-off membrane. Then the filtrate was filtered again through a 10,000 molecular weight cut-off membrane. Three molecular weight fractions can be obtained as a result, a low molecular weight, a middle molecular weight, and a high molecular weight fraction. A block copolymer can then be synthesized by a ring opening polymerization of D,L-lactide from the terminal hydroxyl group of the poly(IPAAm-co-DMAAm) of the middle molecular weight fraction. The resulting poly(IPAAm-co-DMAAm)-b-poly(D,L-lactide) copolymer can be purified as described in Kohori, F. et al. (1999). (Kohori, F. et al., “Control of adriamycin cytotoxic activity using thermally responsive polymeric micelles composed of poly(N-isopropylacrylamide-co-N,N-dimethylacrylamide)-b-poly(D,L-lacide).”, Colloids Surfaces B: Biointerfaces 16: 195-205, (1999)).

The catecholic butanes can be loaded into the inner cores of micelles and the micelles prepared simultaneously by a dialysis method. For example, a chloride salt of the catecholic butanes can be dissolved in N,N-dimethylacetamide (“DMAC”) and added by triethylamine (“TEA”). The poly(IPAAm-co-DMAAm)-b-poly(D,L-lactide) block copolymer can be dissolved in DMAC, and distilled water can be added. The solution of catecholic butanes and the block copolymer solution can be mixed at room temperature, followed by dialysis against distilled water using a dialysis membrane with 12,000-14,000 molecular weight cut-off (Spectra/Por®2, spectrum Medical Indus., CA. U.S.A.) at 25° C. Poly(IPAAm-co-DMAAm)-h-poly(D,L-lactide) micelles incorporating catecholic butanes can be purified by filtration with a 20 nm pore sized microfiltration membrane (ANODISC™, Whatman International), as described in Kohori, F., et al. (1999), supra.

Preparation of Multivesicular Liposomes Containing Catecholic Butanes:

Multivesicular liposomes (“MVL”) can be produced by any method conventional in the art, such as, for example, the double emulsification process as described in Mantriprgada (Mantriprgada, S., “A lipid based depot (DepoFoam® technology) for sustained release drug delivery.”, Prog Lipid Res. 41: 392-406, (2002)). Briefly, in the double emulsification process, a “water-in-oil” emulsion is first made by dissolving amphipathic lipids, such as a phospholipid containing at least one neutral lipid, such as a triglyceride, in one or more volatile organic solvents, and adding to this lipid component an immiscible first aqueous component and a hydrophobic catecholic butane, such as a hydrophobic catecholic butane. The mixture is then emulsified to form a water-in-oil emulsion, and then mixed with a second immiscible aqueous component followed by mechanical mixing to form solvent spherules suspended in the second aqueous component, forming a water-in-oil-in-water emulsion. The solvent spherules will contain multiple aqueous droplets with the catecholic butane dissolved in them. The organic solvent is then removed from the spherules, generally by evaporation, by reduced pressure or by passing a stream of gas over or through the suspension. When the solvent is completely removed, the spherules become MVL, such as DepoFoam particles. When the neutral lipid is omitted in this process, the conventional multilamellar vesicles or unilamellar vesicles will be formed instead of the MVL.

Formulation of Catecholic Butanes for Oral Delivery:

Some catecholic butanes are water-soluble, hydrophilic compounds, such as G₄N. This invention includes formulation of hydrophilic compounds in a pharmaceutically acceptable carrier or excipient and delivery of such as oral formulations, such as in the form of an aqueous liquid solution of the compound, or the compounds can be lyophilized and delivered as a powder, or made into a tablet, or the compounds can be encapsulated.

The tablets herein can be enteric coated tablets. The formulations herein can be sustained release and/or controlled release including either slow release or rapid release formulations.

The amount of the catecholic butanes to be included in the oral formulations can be adjusted depending on the desired dose to be administered to a subject. Such an adjustment is within the skill of persons conventional in the art.

Some catecholic butanes are hydrophobic or lipophilic compounds, such as M₄N. The absorption of lipophilic compounds in the gut can be improved by using pharmaceutically acceptable carriers that can enhance the rate or extent of solubilization of the compound into the aqueous intestinal fluid. Lipidic carriers are known in the art, such as, for example, as described in Stuchlik (Stuchlik, M. and Zak, S., “Lipid-Based Vehicle for Oral Delivery, Biomed. Papers 145(2): 17-26, (2001)). The formulations herein can be delivered as oral liquids or can be encapsulated into various types of capsules.

The present invention includes, in one embodiment, a formulation containing the lipophilic catecholic butanes that are formulated for oral delivery by dissolution of such compounds in triacylglycerols, and the formulation is then encapsulated for oral delivery. Triacyglycerols are molecules with long chain and/or medium chain fatty acids linked to a glycerol molecule. The long chain fatty acids range from about C₁₄ to C₂₄, and can be found in common fat. The medium chain fatty acids range from about C₆ to C₁₂, and can be found in coconut oil or palm kernel oil. Triacylglycerols suitable for use herein include structured lipids that contain mixtures of either short-chain or medium chain fatty acids or both, esterified on the same glycerol molecule.

In another embodiment of the present invention, one or more surfactants can be added to a mixture of catecholic butanes and lipidic carrier such that the drug is present in fine droplets of oil/surfactant mix. The surfactants can act to disperse the oily formulation on dilution in the gastrointestinal fluid.

The present invention also includes a formulation for oral delivery of the catecholic butanes in the form of a micro-emulsion consisting of hydrophilic surfactant and oil. The micro-emulsion particles can be surfactant micelles containing solubilized oil and drug.

Also suitable for oral administration are formulations of the catecholic butanes in a solid lipid nanoparticle preparation. Solid lipid nanoparticles can be prepared in any manner conventional in the art, such as, for example, as described in Stuchlik, M. and Zak, S. (2001), supra.

In one embodiment, the solid lipid nanoparticle can be prepared in a hot homogenization process by homogenization of melted lipids at elevated temperature. In this process, the solid lipid is melted and the catecholic butane is dissolved in the melted lipid. A pre-heated dispersion medium is then mixed with the drug-loaded lipid melt, and the combination is mixed with a homogenisator to form a coarse pre-emulsion. High pressure homogenization is then performed at a temperature above the lipids melting point to produce a oil/water-nanoemulsion. The nanoemulsion is cooled down to room temperature to form solid lipid nanoparticles.

In another embodiment of the present invention, the solid lipid nanoparticles can be prepared in a cold homogenization process. In this process, the lipid is melted and the catecholic butane is dissolved in the melted lipid. The drug-loaded lipid is then solidified in liquid nitrogen or dry ice. The solid drug-lipid is ground in a powder mill to form 50-100 μm particles. The lipid particles are then dispersed in cold aqueous dispersion medium and homogenized at room temperature or below to form solid lipid nanoparticles.

The present invention also includes formulation of the lipophilic catecholic butanes in liposomes or micelles for oral delivery. These formulations can be made in any manner conventional in the art. Micelles are typically lipid monolayer vesicles in which the hydrophobic drug associates with the hydrophobic regions on the monolayer. Liposomes are typically phospholipids bilayer vesicles. The lipophilic catecholic butane will typically reside in the center of these vesicles.

Additional suitable formulations of catecholic butanes for oral delivery in accordance with the present invention are described in U.S. Provisional Patent Application Ser. No. 60/647,495 filed on Jan. 27, 2005, and International Application filed Jan. 27, 2006, entitled “Oral Formulations for Deliver of Catecholic Butanes, Including NDGA Compounds,” Attorney Docket No. 682714-9WO, the entire contents of which are incorporated herein by reference.

Formulation of Catecholic Butanes for Intranasal Delivery:

The present invention includes formulations of catecholic butanes for intranasal delivery and intranasal delivery thereof. Intranasal delivery may advantageously build up a higher concentration of the active agents in the brain than can be achieved by intravenous administration. Also, this mode of delivery avoids the problem of first pass metabolism in the liver and gut of the subject receiving the drug.

The amount of the active agents that can be absorbed partly depends on the solubility of the drug in the mucus, a composition that consists of about 95% water solution of serum proteins, glycoproteins, lipids and electrolytes. Generally, as lipophilicity of the active agents herein increases, the drug concentration in the CSF also increases. See, for example, (Minn, A. et al., “Drug transport into the mammalian brain: the nasal pathway and its specific metabolic barrier.”, J. Drug Target, 10: 285-296, (2002)).

The hydrophilic catecholic butanes can be dissolved in a pharmaceutically acceptable carrier such as saline, phosphate buffer, or phosphate buffered saline. In one embodiment, a 0.05 M phosphate buffer at pH 7.4 can be used as the carrier, as described in, for example, Kao, et al. (Kao, H. D. et al., “Enhancement of the Systemic and CNS Specific Delivery of L-Dopa by the Nasal Administration of Its Water Soluble Prodrugs,”, Pharmaceut. Res., 17(8): 978-984, (2000)).

Intranasal delivery of the present agents may be optimized by adjusting the position of the subject when administering the agents. For example, the head of the patient may be variously positioned upright-90°, supine-90°, supine-45°, or supine-70°, to obtain maximal effect.

The carrier of the composition of catecholic butanes may be any material that is pharmaceutically acceptable and compatible with the active agents of the composition. Where the carrier is a liquid, it can be hypotonic or isotonic with nasal fluids and within the pH of about 4.5 to about 7.5. Where the carrier is in powdered form it is also within an acceptable pH range.

The carrier composition for intranasal delivery may optionally contain lipophilic substances that may enhance absorption of the active agents across the nasal membrane and into the brain via the olfactory neural pathway. Examples of such lipophilic substances include, but are not limited to, gangliosides and phosphatidylserine. One or several lipophilic adjuvants may be included in the composition, such as, in the form of micelles.

The pharmaceutical composition of active agents for intranasal delivery to a subject for treatment of influenza can be formulated in the manner conventional in the art as described in, for example, U.S. Pat. No. 6,180,603. For example, the composition herein can be formulated as a powder, granules, solution, aerosol, drops, nanoparticles, or liposomes. In addition to the active agents, the composition may contain appropriate adjuvants, buffers, preservatives, salts. Solutions such as nose drops may contain anti-oxidants, buffers, and the like.

Delivery by Implantation:

The catecholic butanes herein may be delivered to a subject for treatment by surgical implantation, such as subcutaneous implantation of a biodegradable polymer containing the catecholic butanes. This treatment may be combined with other conventional therapy besides or in addition to surgery.

Thus, the biodegradable polymer herein can be any polymer or copolymer that would dissolve in the interstitial fluid, without any toxicity or adverse effect on host tissues. Preferably, the polymer or monomers from which the polymer is synthesized is approved by the Food and Drug Administration for administration into humans. A copolymer having monomers of different dissolution properties is preferred so as to control the dynamics of degradation, such as increasing the proportion of one monomer over the other to control rate of dissolution.

In one embodiment, the polymer is a copolymer of 1,3-bis-(p-carboxyphenoxy)propane and sebacic acid [p(CPP:SA)], as described in Fleming A. B. and Saltzman, W. M., Pharmacokinetics of the Carmustine Implant, Clin. Pharmacokinet, 41(6): 403-419 (2002); and Brem, H., and Gabikian, P., “Biodegradable polymer implants to treat brain tumors.”, J. Control. Rel. 74: 63-67, (2001)). In another embodiment, the polymer is a copolymer of polyethylene glycol (“PEG”) and sebacic acid, as described in Fu, et al. (Fu, J. et al., “New Polymeric Carriers for Controlled Drug Delivery Following Inhalation or Injection.”, Biomaterials, 23: 4425-4433, (2002)).

Polymer delivery systems are applicable to delivery of both hydrophobic and hydrophilic catecholic butanes herein. The catecholic butanes are combined with the biodegradable polymers and surgically implanted. Some polymer compositions are also usable for intravenous or inhalation therapy herein.

Delivery Through Inhalation

The catecholic butanes herein may be delivered systemically and/or locally by administration to the lungs through inhalation. Inhalation delivery of drugs has been well accepted as a method of achieving high drug concentration in the pulmonary tissues without triggering substantial systemic toxicity, as well as a method of accomplishing systemic circulation of the drug. The techniques for producing such formulations are conventional in the art. Efficacy against pulmonary diseases may be seen with either hydrophobic or hydrophilic catecholic butanes delivered in this manner.

For pulmonary delivery via inhalation, the catecholic butanes herein may be formulated into dry powders, aqueous solutions, liposomes, nanoparticles, or polymers and administered, for example, as aerosols. Hydrophilic formulations may also be taken up through the alveolar surfaces and into the bloodstream for systemic applications.

In one embodiment, the polymers containing the active agents herein are made and used as described in Fu, J. et al. (2002), supra. For example, the polymers herein can be polymers of sebacic acid and polyethylene glycol (“PEG”), or can be poly(lactic-co-glycolic) acid (“PLGA”), or polymers of polyethyleneimine (“PEI”) and poly-L-lysine (“PLL”).

In another embodiment, the catecholic butanes for inhalation delivery may be dissolved in saline or ethanol before nebulization and administered, as described in Choi, et al. (Choi, W. S. et al., “Inhalation delivery of proteins from ethanol suspensions.”, Proc. Natl. Acad. Sci. USA, 98(20): 11103-11107, (2001)).

In a further embodiment, the agents herein are also effective when delivered as a dry powder, prepared in the manner conventional in the art, as described in, for example, Patton, et al. (Patton, J. S. et al., “Inhaled Insulin,”, Adv. Drug Deliv. Rev., 35: 235-247 (1999) (2001)).

The present invention includes delivery of the catecholic butanes with the aid of microprocessors embedded into drug delivery devices, such as, for example, SmartMist™ and AERx™, as described in, for example, Gonda, I. et al. (1998), “Inhalation delivery systems with compliance and disease management capabilities.” J. Control. Rel. 53: 269-274.

The catecholic butanes and compositions of the present invention are administered to treat any influenza viral infection. In certain preferred embodiments, the influenza strain to be treated is an avain strain. In certain preferred embodiments, the influenza infection to be treated is based on an H5N1 avian strain. In certain preferred embodiments of the present invention, the catecholic butanes and compositions are administered to a human subject infected with an avian strain of influenza. Additionally, in certain preferred embodiments, the catecholic butanes and compositions are administered to a human subject suffering from a combination of human and avian influenza infections. After reading the present disclosure, those skilled in the art will recognize other disease states and/or symptoms which might be treated and/or mitigated by the administration of formulations of the present invention.

While not being bound to any particular theory of influenza pathogenesis or symptomatic response, it is believed that influenza viral infection in humans induces proinflammatory cytokine dysregulation. The clinical features of severe human H5N1 disease are compatible with virus-induced cytokine dysregulation. While all influenza viral infections are believed to induce proinflammatory cytokines, the HSN1/97 viruses induced much higher gene transcription of proinflammatory cytokines than human influenza A virus subtypes H3N2 or H1N1 (Cheung C Y, Poon L L, Lau A S, et al., “Induction of proinflammatory cytokines in human macrophages by influenza A (H5N1) viruses: a mechanism for the unusual severity of human disease?” Lancet, 2002 360(9348): 1831-7). Cytokines particularly induced were TNF-α (also referred to herein and in the Figs. as “TNF”) and interferon beta in human primary monocyte-derived macrophages in vitro (Id.).

Influenza viral infections often exhibit severe cold-like symptoms and can often lead to respiratory disorders and/or lethal pneumonia. Patients infected with the H5N1 influenza subtype have had a primary viral pneumonia complicated by syndromes of acute respiratory distress and multiple organ dysfunctions. Lymphopenia and hemophagocytosis have been notable findings in some of these patients. Hemophagocytosis and the syndromes of acute respiratory distress and multiple organ dysfunctions are commonly associated with cytokine dysregulation. Post-mortem reports of H5N1-related deaths in 1997 describe reactive hemophagocytic syndrome with elevated concentrations of the inflammatory cytokines IL-6, IFN-γ and TNF-α. There are many diseases or disorders associated with influenza infection, including, but not limited to, asthma, pneumonia, post-influenza encephalitis, bacterial myositis, changes in cardiac electrocardiogram, bronchitis, tuberculosis, carcinoma, rheumatoid arthritis, osteoarthritis, scleroderma, systemic lupus erythematosis, cystic fibrosis, cachexia, generalized muscle weakness disorders, cardiac failure, Parkinsons Disease, amyotrophic lateral sclerosis or Guillain-Barre syndrome.

Human H5N1 viruses of 2003, like the human H5N1/97 isolates, were shown to have induced the overproduction of proinflammatory cytokines by human monocyte-derived macrophages in vitro. TNF-α is highly induced in primary human macrophages by H5N1 viruses from poultry with similar genotypes to the human viruses (Guan Y, Poon L L, Cheung C Y, et al., “H5N1 influenza: a protean pandemic threat.” Proc Natl Acad Sci USA, 2004 101(21): 8156-61). Accordingly, it is believed that the increased levels of TNF-α and other cytokines from macrophages is relevant to the severity of illness in patients with influenza A infection, particularly the unusual clinical presentation and severity of illness in patients with H5N1 “avian flu”. The systemic inflammatory response, multiorgan dysfunction, and acute respiratory distress syndrome, reactive haemophagocytosis, and lymphopenia were distinctive features in patients with severe H5N1 disease.

TNF-α is well known for its ability to induce apoptosis. Apoptosis-inducing activity may also contribute to influenza pathogenesis since apoptosis is critical for efficient influenza virus replication. Efficient replication of both human and avian influenza viruses has been linked to upregulation of TNF superfamily members TRAIL and FasL (Wurzer W J, Ehrhardt C, Pleschka S, et al. “NF-kappaB-dependent induction of tumor necrosis factor-related apoptosis-inducing ligand (TRAIL) and Fas/FasL is crucial for efficient influenza virus propagation.” J Biol Chem, 2004 279(30): 30931-7).

Again, while not being bound by any particular theory, it has been suggested and it is believed that elevated production of inflammatory cytokines in response to influenza virus genomic RNA is signaled by Toll-like receptors on the membranes of cells of the immune system (Diebold S S, Kaisho T, Hemmi H, et al. “Innate antiviral responses by means of TLR7-mediated recognition of single-stranded RNA.” Science, 2004 303(5663): 1529-31). Stimulation of macrophages with lipopolysaccharide (“LPS”), a bacterial endotoxin, also results in the production of pro-inflammatory cytokines such as TNFα, IL-1, IL-6, IL-10 and pro-inflammatory lipid mediators such as prostaglandins, leukotrienes and platelet-activating factor. Cytokine production in response to LPS has been demonstrated to act through the Toll-like receptor pathways, similar to the influenza virus response (Takeda K, Kaisho T, and Akira S. “Toll-like receptors.” Annu Rev Immunol, 2003 21: 335-76).

Catecholic butanes of the general formulas (I), (II) and (III), such as, for example, M₄N or G₄N, and compositions containing one or more of the catecholic butanes according to the present invention inhibits the production of TNF-α and other pro-inflammatory cytokines, and prostaglandin E₂ and other pro-inflammatory lipid mediators, in response to stimulation of LPS or viral infection in murine monocyte-derived macrophages. Murine monocyte-derived macrophage cell line (RAW 264.7) highly induces TNF-α production in response to LPS comparable to primary human macrophages, and thus represents a suitable model to predict human drug effects in the TNF system.

The invention will now be described in more detail with reference to the following non-limiting examples.

### Example 1

The effects of administering a catecholic butane of the general formula (I), namely M₄N, on the production of TNF-α by LPS-stimulated RAW 264.7 macrophages were investigated to determine the ability of M₄N to inhibit TNF-α induction. Methods similar to that of this Example can be used to determine the effect of any catecholic butane of the general formula (I) on the production of any proinflammatory cytokine in any LPS-stimulated macrophage cell.

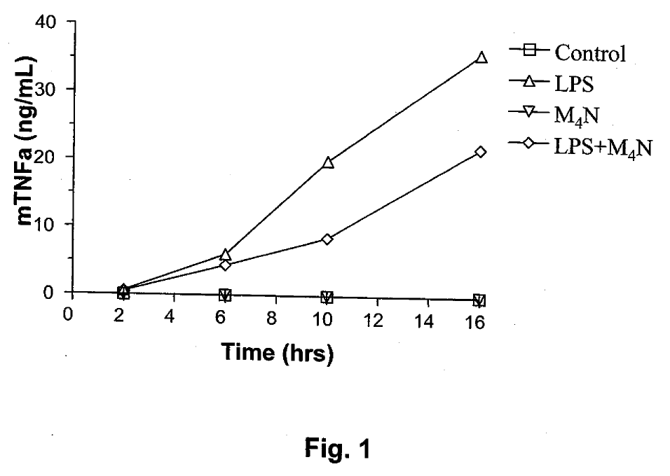

As shown in FIG. 1 and explained below, M₄N inhibits the LPS-induced TNF-α overexpression in RAW 264.7 macrophages with inhibition maximal at 57% at 10 hours post induction.

More specifically regarding the method used to determine the ability of M₄N to inhibit TNF-α induction by LPS, 1.5×10⁵ macrophages were either left untreated (control) or cultured for the indicated times with LPS (1 μg/ml), M₄N (25 μM), or both compounds. RAW 264.7 cells are mouse monocyte macrophages. The LPS used was from Salmonella minnesota R595 and is available from List Biological Laboratories, Inc. (Campbell, Calif.). Levels of TNF-α in culture supernatants were then determined using a mouse TNF-α specific immunoassay by interpolation from a standard curve. All measurements were performed in duplicate and in each case error bars were smaller than symbol size.

RAW264.7 macrophages were purchased from the ATCC and cultured in Dulbecco's modified Eagles medium supplemented with 10% fetal bovine serum (FBS) and maintained at 37° C. in 8% carbon dioxide. FBS was purchased from Atlanta Biologicals (Atlanta, Ga.) while all other media components were purchased from Sigma Aldrich (St. Louis, Mo.). For TNF production, cells were harvested by trypsinization, centrifuged, counted and 1.5×10⁵ of cells were plated in 24 well tissue culture plates and cultured overnight. The cells were then stimulated for the times indicated in FIG. 1 with one microgram per milliliter of lipopolysaccharide (LPS) in the absence/presence of 25 μM M₄N. LPS was dissolved in tissue culture media and sonicated prior to addition to the wells. M₄N stock solutions were prepared in DMSO, then diluted in culture media prior to addition to the wells. Resulting supernatants were collected, centrifuged at 8,000 rpm for two minutes to remove cells and debris and stored at −20° C. Levels of TNF-α in culture supernatants were determined using the Quantikine mouse TNF-α/TNFSF1A immunoassay purchased from R&D Systems Inc. (Minneapolis, Minn.). The assay is a sandwich style capture ELISA. Wells were supplied pre-coated with an affinity purified polyclonal antibody specific for murine TNF-α. Supernatants were added to the wells, incubated, and any TNF-α present was captured by the immobilized antibody. Following washing, an enzyme linked anti-TNF-α-antibody was added and a second incubation step was performed. The wells were washed again and a substrate solution was added. Cleavage of the substrate produces a blue solution which then turns yellow upon addition of the stop solution. Color intensity was then determined at 450 nm using a BMG POLARstar galaxy microplate reader. Standard solutions of recombinant murine TNF-α are supplied by the manufacturer to produce a standard curve and levels of TNF-α in culture supernatants were determined by interpolation from the standard curve. All points shown in FIG. 1 were performed in duplicate and mean values were used for quantitation.

Inhibition of TNF-α production by M₄N was not observed in RAW 264.7 macrophages following induction with phorbol myristyl acetate (PMA) or A23187 (calcium ionophore), rather than LPS. PMA and A23187 are believed to work non-specifically, independent of cell surface receptors and most signal transduction processes. Therefore, while not being bound to any particular theory, the ability of M₄N to inhibit the LPS-induced production of TNF-α may stem from effects on the upstream signaling and activating phase of the TNF response, rather than on the downstream processes responsible for synthesis and release of TNF-α. Thus, it is believed that the beneficial effects of M₄N treatment for the treatment of H5N1 infection may be observed without causing a potentially harmful nonspecific drop in TNF-α production.

Results from this Example demonstrate that a catecholic butane of the general formula (I) can inhibit the over production of TNF-α in response to LPS stimulation, indicating that the compound and related catecholic butanes and NDGA derivatives can be used to treat diseases or disorders mediated by increased levels of TNF-α upon influenza infection.

### Example 2

The effects of administering a catecholic butane of the general formula (I), namely M₄N, on TNF-α-induced apoptosis in murine fibroblasts were investigated to determine the ability of M₄N to inhibit TNF-α-induced apoptosis. Methods similar to that of this Example can be used to determine the effect of any catecholic butane of the general formula (I) on TNF-α-induced apoptosis in any type of cells.

Influenza infection induces production of TNF-α, and TNF-α is well known for its pro-apoptotic activity. Influenza requires apoptosis for efficient replication and blocking TNF-α-induced apoptosis may reduce influenza replication and disease.

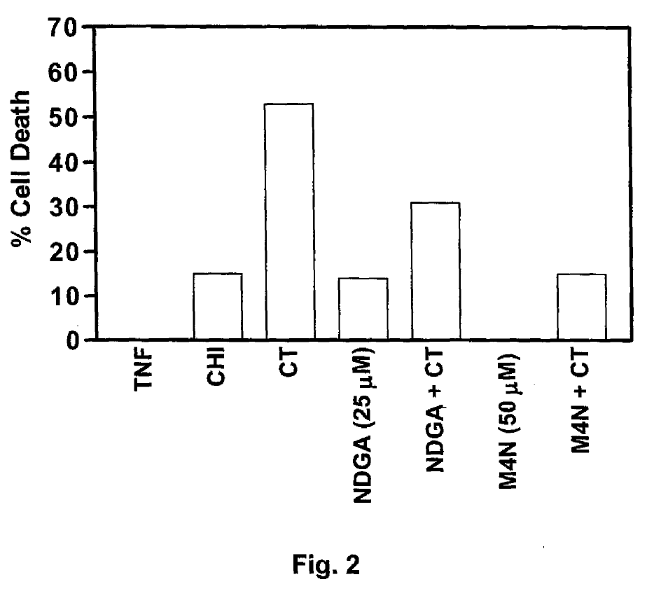

As shown in FIG. 2 and explained below, M₄N strongly inhibits TNF-α-induced apoptosis in cells rendered sensitive to TNF by cycloheximide. C3HA murine fibroblasts were incubated with human recombinant TNF-α (20 ng/ml), cycloheximide (CHI) (10 μg/ml), or both, in the absence/presence of NDGA (25 μM) or M₄N (50 μM). All compounds were added simultaneously and treatments were for 6 hours. Rhodamine 123 was added during the last half hour and fluorescence measured using a BMG POLARstar galaxy flourimeter.

More specifically, C3HA cells were cultured in Dulbecco's modified Eagles medium supplemented with 10% fetal bovine serum and maintained at 37° C. in 8% carbon dioxide. The C3HA cell line is a 3T3-like murine fibroblast cell line developed from C3H mice. Fetal bovine serum was purchased from Atlanta Biologicals and all other media components were purchased from Sigma Aldrich. For apoptosis assays, cells were harvested by trypsinization, centrifuged, counted and 1.5×10⁴ cells added to each well of a flat bottom 96 well microtiter plate in culture media. Cells were allowed to adhere at least six (6) hours prior to the addition to TNF (20 ng/ml), CHI (10 μg/milliliter), or both, in the absence/presence of nordihydroguaiaretic acid (25 μM) or M₄N (50 μM). Human recombinant TNF-α was purchased from Peprotech (Rocky Hill, N.J.) while CHI was purchased from EMD Biosciences Inc. (San Diego, Calif.). Both were dissolved in culture media. NDGA was also dissolved in culture media while M₄N stock solutions were prepared in DMSO then diluted in culture media prior to addition to the wells. All compounds were added simultaneously in a total volume of 200 microliters and allowed to incubate for six (6) hours. During the last thirty (30) minutes of the incubation period, 50 μL of Rhodamine 123 was added to each well to final concentration of 2 μg per milliliter. Rhodamine 123 was purchased from Molecular Probes Inc. (Eugene, Oreg.) and was diluted in culture media. Rhodamine 123 is sequestered by energized mitochondria and live healthy cells display strong mitochondrial fluorescence. In contrast, fluorescence is decreased in cells undergoing apoptosis since apoptotic cells often undergo mitochondrial permeability transition and the mitochondria lose their membrane potential. As a result, Rhodamine 123 fluorescence is dramatically decreased in apoptotic cells. Fluorescence intensity was then determined using a BMG POLARstar galaxy microplate reader with excitation and emission wavelengths set at 492 and 538 nanometers, respectively. All points were performed in triplicate and percentage cell death was calculated from the following formula:

[(control−experimental)/control]*100

Results from this Example demonstrate that a catecholic butane of the general formula (I) can inhibit the TNF-α-induced apoptosis in a cell, indicating that the compound and related catecholic butanes and NDGA derivatives can be used to reduce influenza replication in a host cell, which requires apoptosis for efficient replication.

### Example 3

The effects of administering a catecholic butane of the general formula (I), namely M₄N, on the production of prostaglandin E₂ (“PGE₂”), prostaglandin F_{1α} (“PGF_{1α}”) and prostaglandin F_{2α} (“PGF_{2α}”), by LPS-induced RAW 264.7 macrophages were investigated to determine the ability of M₄N to inhibit over production of prostaglandins in response to influenza viral infection. Methods similar to that of this Example can be used to determine the effect of any catecholic butane of the general formula (I) on the production of any pro-inflammatory lipid mediator in any LPS-stimulated macrophage cell.

Prostaglandins are autocrine and paracrine lipid mediators found in virtually all tissues and organs. They are synthesized in the cell from the essential fatty acids, such as the gamma-linolenic acid, arachidonic acid, and eicosapentaenoic acid. They act upon a variety of cells, such as platelet cells causing aggregation or disaggregation, vascular smooth muscle cells causing constriction or dilation, spinal neurons causing pain, in addition to endothelium cells, uterine and mast cells, etc. Prostaglandins have a wide variety of actions, including, but not limited to muscular constriction and mediate inflammation. Other effects include calcium movement, hormone regulation and cell growth control.

Prostaglandin E₂ is generated from the action of prostaglandin E synthases on prostaglandin H₂ (PGH₂), which is derived from fatty acid via the action of cyclooxygenases (COX-1 and COX-2). PGE₂ is induced during scenarios of influenza infection. Infection with human influenza virus subtype H3N2 increases PGE₂ release in bronchial epithelial cells (Mizumura K, Hashimoto S, Maruoka S, et al., “Role of mitogen-activated protein kinases in influenza virus induction of prostaglandin E₂ from arachidonic acid in bronchial epithelial cells.” Clin Exp Allergy, 2003 33(9): 1244-51).

PGF_{2α} can be produced by three pathways from three distinct substrates including PGH₂, PGE₂, or PGD₂. PGF_{2α} causes smooth muscle contraction and its activity has been linked to asthma and parturition.

Prostacyclin, also known as PGI₂, is generated from PGH₂ by the action of PGI synthase, which is widely expressed by many cell types. Prostacyclin is a potent vasodilator and smooth muscle relaxant important in diverse biological responses such as inflammation and parturition. Prostacyclin is unstable, however, and reliable measurements are obtained typically by measuring a stable derivative of prostacyclin known as PGF_{1α} (6-keto-PGF1_{α}.

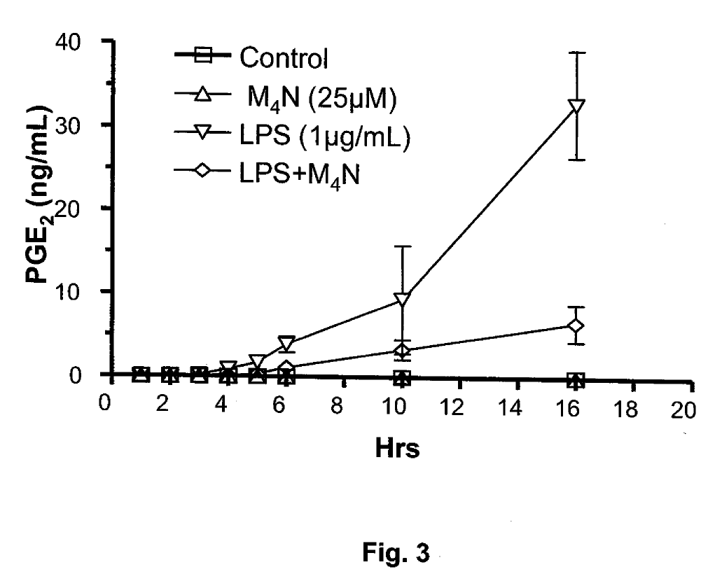

As shown in FIG. 3 and explained below, M₄N has a strong inhibitory effect on LPS-induced PGE₂ production. In FIG. 3, M₄N (25 μM) displayed strong inhibition of LPS-induced production of PGE₂ in RAW 246.7 macrophages. The macrophages were treated with LPS (1 μg/ml) alone or in combination with 25 μM M₄N for the indicated time periods. Supernatants were then assayed for PGE₂ using the prostaglandin E₂ immunoassay (R&D Systems, Minneapolis Minn.). Data shown are means+/−SEM of 2-4 determinations at each time point. The levels of suppression were 72, 64, and 80% at 6, 10, and 16 hours, respectively. The suppressive effects of M₄N persisted in culture throughout the 16 hour time course.

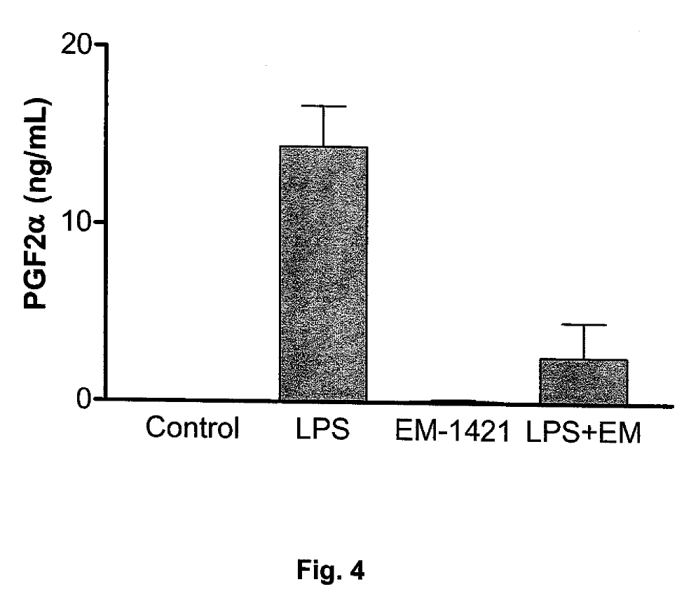

As shown in FIG. 4 and explained below, M₄N also has a strong inhibitory effect on LPS-induced PGF_{2α} production. In FIG. 4, 15 ng/ml of PGF_{2α} was detected from the RAW 264.7 macrophage culture supernatant following 16 h stimulation with LPS (1 μg/ml). The production of PGF_{2α} was inhibited when RAW 264.7 macrophages were treated simultaneously with LPS (1 μg/ml) and M₄N (25 μM) for 16 h. The mean % suppression by M₄N (25 μM) from two experiments was 82%. Levels of PGF_{2α} in RAW 264.7 macrophage culture supernatants were determined by ELISA using the PGF_{2α} ELISA kit (Assay Designs, Ann Arbor, Mich.). Data shown are means+/−SEM from two independent experiments.

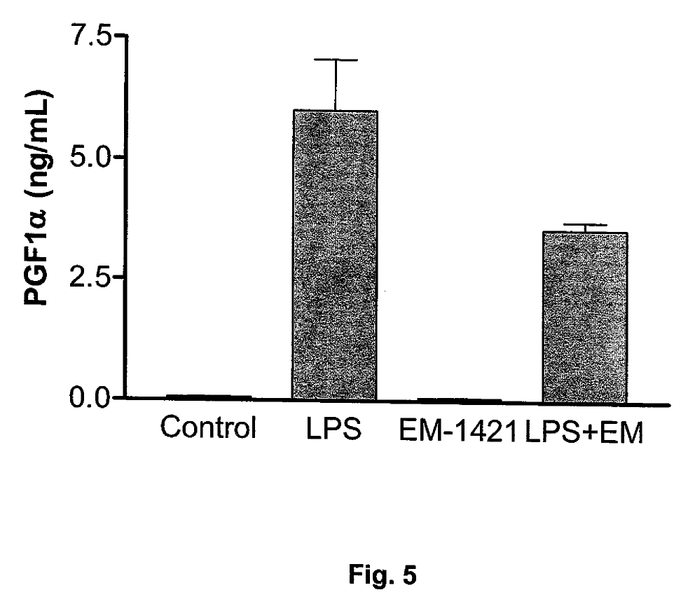

As shown in FIG. 5 and explained below, M₄N has some inhibitory effect on LPS-induced PGF_{1α} (production. In FIG. 5, 5-6 ng/ml of PGF_{1α} was detected from the RAW 264.7 macrophage culture supernatant following 16 h stimulation with LPS (1 μg/ml). The production of PGF_{1α} was inhibited when RAW 264.7 macrophages were treated simultaneously with LPS (1 μg/ml) and M₄N (25 μM) for 16 h. The mean % suppression from two experiments was 41%. Levels of PGF_{1α} in RAW 264.7 macrophage culture supernatants were determined by ELISA using the PGF_{1α} ELISA kit (R&D Systems, Minneapolis Minn.). Data shown are means+/−SEM from two independent experiments.

To confirm that the relatively modest inhibition of the production of PGF_{1α} (an indicator of PGI₂/prostacyclin) shown in FIG. 5 was not due to some experimental errors, the % suppression of the production of PGE₂ in these RAW 264.7 macrophage culture supernatants was also measured. Consistent with the result in FIG. 3, more than 90% suppression of PGE₂ in these supernatants was detected, suggesting that the observed modest inhibition of the production of PGF_{1α} was unlikely due to experimental errors related to the cells, LPS, and M₄N.

Because M₄N, also called EM-1421, exerts strong inhibitory effects on prostaglandin and leukotriene production, it can be particularly well suited for treating inflammatory conditions in the lung, such as asthma, caused by influenza infection, which tend to be dependent on the lipid mediators. The PGI synthases, as opposed to the PGE and PGF synthases may be relatively resistant to EM-1421 accounting for the substantial production of PGF_{1α} in the presence of EM-1421.

More specifically regarding the method used to determine the effect of M₄N on the production of PGE₂, RAW264.7 macrophages were purchased and cultured and maintained in accordance with the procedure set forth in Example 1. Prostaglandin E₂ production in the cells was accomplished by harvesting the cells by trypsinization, and centrifugation. The cells were counted and 1.5×10⁵ cells were plated in 24 well tissue culture plates and cultured overnight. The cells were then stimulated for the times indicated in FIG. 5 with one μg per milliliter of LPS in the absence/presence of 25 μM M₄N. LPS was dissolved in tissue culture media and sonicated prior to the addition to the wells. M₄N stop solutions were prepared in DMSO and then diluted in culture media prior to addition to the wells. The resulting supernatants were collected, centrifuged at 8,000 rpms for 2 minutes to remove cells and debris and stored at −20° C. Levels of prostaglandin E₂ in culture supernatants were determined using the prostaglandin E₂ immunoassay purchased from R&D Systems Inc. The assay is a competition type ELISA. Prostaglandin E₂ present in supernatants competes with a fixed amount of alkaline phosphatase-labeled prostaglandin E₂ for binding to a mouse monoclonal anti-prostaglandin E₂ antibody. The resulting complex is bound by a goat anti-mouse antibody supplied bound to microtiter wells. Following washing, a color producing substrate is added to quantitate the amount of bound enzyme. Color intensity was determined at 405 nanometers using a BMG POLARstar galaxy microplate reader. Standard solutions of prostaglandin E₂ are supplied by the manufacturer to produce a standard curve and levels of prostaglandin E₂ in culture supernatants were determined by interpolation from the standard curve. All points were performed in duplicate and mean values used for quantitation.

Similar methods were used to determine the effect of M₄N on the production of PGF_{1α} and PGF_{2α}.

Results from this Example demonstrate that a catecholic butane of the general formula (I) can inhibit the over production of prostaglandins in response to LPS stimulation, indicating that the compound and related catecholic butanes and NDGA derivatives can be used to treat diseases or disorders mediated by increased levels of prostaglandins upon influenza infection.

### Example 4

The effects of administering a catecholic butane of the general formula (I), namely M₄N, on the production of a group of cytokines by RAW 264.7 macrophages were investigated to determine the induction of the cytokines by LPS stimulation and the ability of M₄N to inhibit the induction.

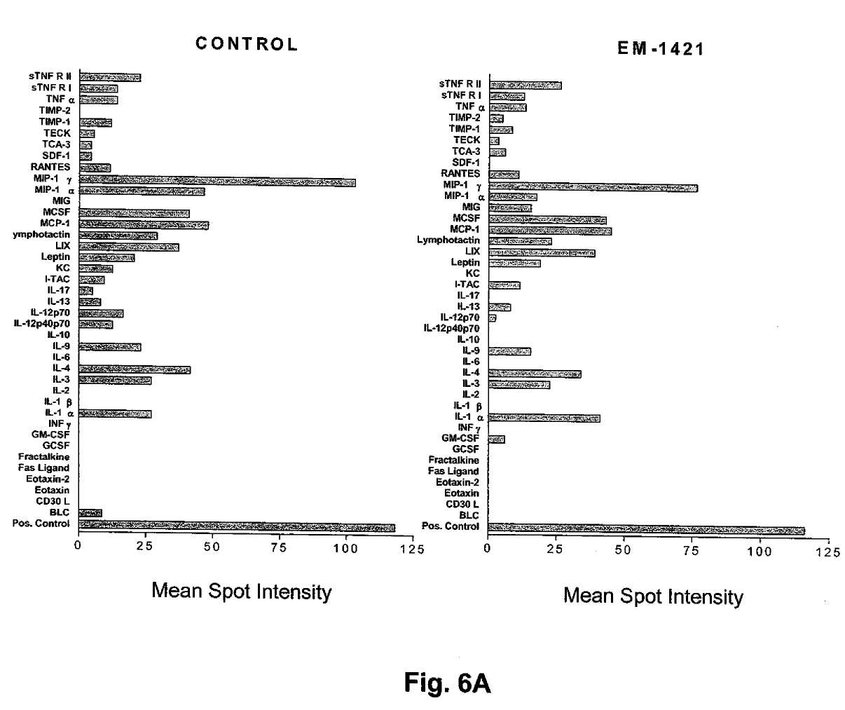

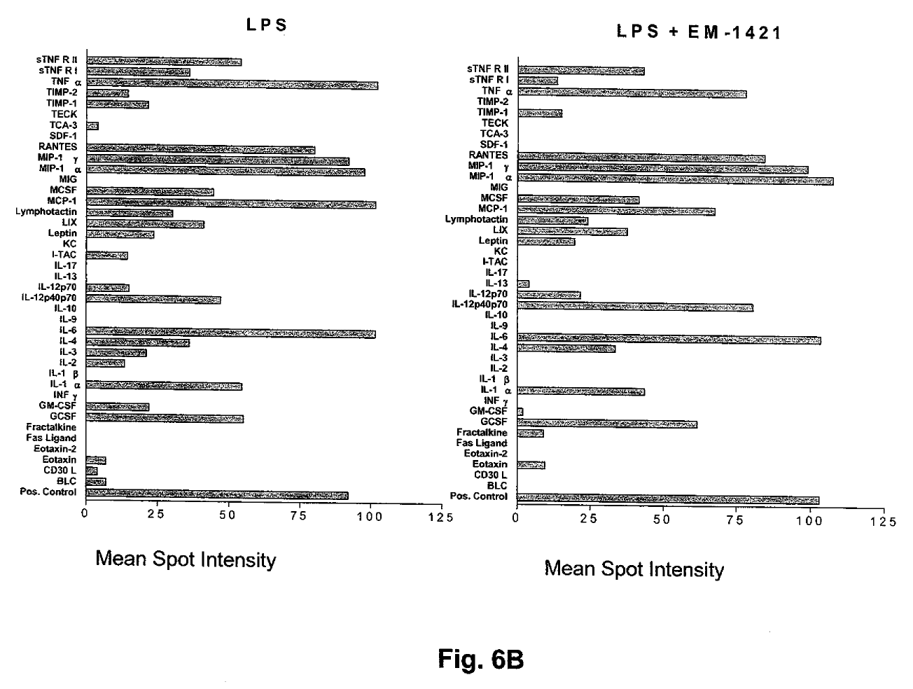

Antibody (“Ab”) array technology was used in this study. As shown in FIG. 6 and explained below, M₄N has an inhibitory effect on LPS-induced production of several cytokines. A number of cytokines were detected in supernatants from RAW264.7 macrophages without the stimulation of LPS or the treatment of EM-1421 (“Control” panel in FIG. 6A), although levels of cytokine production were generally low. Overall, this pattern of cytokine production was retained following treatment with EM-1421 at 25 μM final concentration (“EM-1421” panel in FIG. 6A), although production of several cytokines was reduced (KC, BLC, IL-4, IL-9, MIP-1α, MIP-1γ, and IL-12p40p70) and the production of two cytokines was increased (IL-1α and MIG). This reinforces the clinical findings that EM-1421 is safe.

LPS caused substantial increases (>20%) in the production of many cytokines (“LPS” panel in FIG. 6B), including, RANTES, IL-1α, IL-2, TIMP-1, TIMP-2, TNF-α, IL-6, MCP-1, sTNFRI, sTNFRII, IL-12p40, MIP-1α, and G-CSF. In several cases, these increases were either partially or completely offset by EM-1421 (“LPS+EM-1421” panel in FIG. 6B). Among cytokines produced at high levels, EM-1421 inhibited LPS-induced production of IL-1α by about 20%, TNF-α by about 24%, MCP-1 by about 33%, sTNFRI by about 63%, and sTNFRII by about 20%. Among cytokines produced at low levels, EM-1421 inhibited the LPS-induced production of I-TAC by about 100%, IL-2 by about 100%, TIMP-1 by about 30%, TIMP-2 by about 100%, BLC by about 100%, and IL-3 by about 100%. However, because these cytokines were produced at low levels it is difficult to predict the significance of these observations. Interestingly, EM-1421 did not inhibit the LPS-induced production of RANTES, IL-6, IL-12p70, MIP1-α, and G-CSF, and increased the LPS-induced production of IL-12p40p70 by about 43%.

More specifically regarding the method used to determine the effect of M₄N on the production of cytokines, “mouse inflammatory array-1” (RayBiotech, Inc., Atlanta, Ga.) was used in the method. RAW264.7 macrophages were purchased, cultured, stimulated with LPS (1 μg/ml), treated with EM-1421 (25 μM), and harvested in accordance with the procedure set forth in Example 1. Cytokines in culture supernatants of RAW264.7 macrophage with or without stimulation of LPS (1 μg/ml) and with or without treatment of EM-1421 (25 μM) were measured using the mouse inflammatory array-1 according to the manufacturer's instruction. Briefly, culture supernatants were incubated with nitrocellulose Ab arrays for about 2 h, washed, exposed to secondary Ab solution, developed with ECL solution and exposed to X-ray film. Array autoradiographs were scanned. Photoshop (Adobe) was used to analyze and determine mean pixel intensity for each array position. Mean values for duplicate spots are plotted in FIGS. 6A and 6B. Positive control spots were about 100 units on each array and SEM was less than 10% for all duplicate spots, and for the vast majority of the duplicate spots, SEM was less than 1%.

Results from this Example demonstrate that a catecholic butane of the general formula (I) can inhibit the over production of several other cytokines in addition to TNF-α in response to LPS stimulation, indicating that the compound and related catecholic butanes and NDGA derivatives can be used to treat diseases or disorders mediated by increased levels of the cytokines upon influenza infection.

### Example 5

The effects of administering a catecholic butane of the general formula (I), namely M₄N, on the production of influenza strain A/WS/33 from MDCK cells and RAW 264.7 macrophages were investigated to determine the ability of M₄N to inhibit the growth of the influenza virus in these cells. Methods similar to that of this Example can be used to determine the effect of any catecholic butane of the general formula (I) on the growth or replication of any strain of influenza virus in any type of cells.

A/WS/33 is a strain of influenza A virus commercially available from American Type Culture Collection (ATCC) (Manassas, Va.). It was isolated from a patient with influenza. Recommended hosts for A/WS/33 include chicken embryo, ferrets, and mouse.

MDCK cells are epithelial-like cells derived from a kidney of an apparently normal adult female cocker spaniel. They were shown to support the growth of various types of virus, including influenza A virus. MDCK cells were used to produce high titer stocks of A/WS/33 and for quantitative assays to measure amounts of infectious virus in culture supernatants from the experiments. It was determined that 25 μM was the highest concentration of EM-1421 that could be used in MDCK cells without causing toxic effects. A variety of quantitative assays were established to monitor A/WS/33 replication, including, but not limited to, cytopathicity (TCID₅₀), plaque, immunofocus, and immunofluorescence.

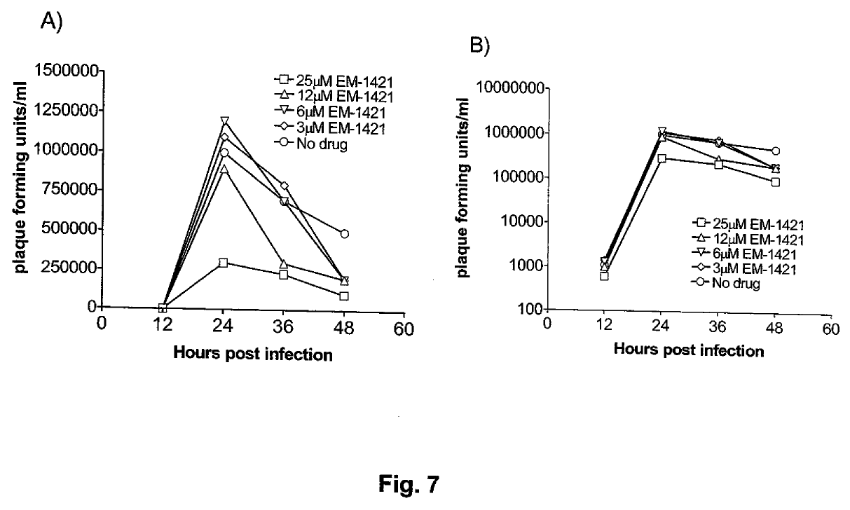

As shown in FIG. 7, where panels A and B display the same data but with linear and log y-axes, respectively, EM-1421 at a concentration of 25 μM inhibited A/WS/33 replication in MDCK cells by appx. 75%. The log plot, which is used typically to visualize large differences in virus titers, shows that the inhibitory effect of EM-1421 was less than 1 log unit.

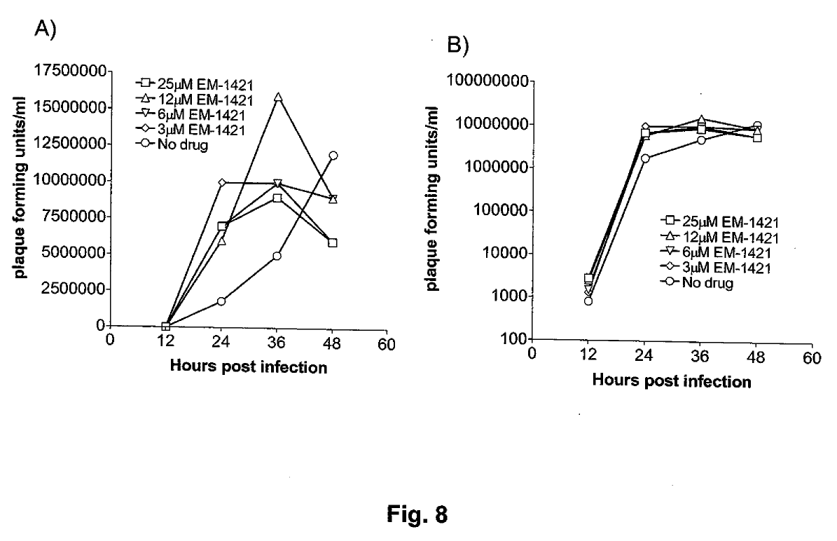

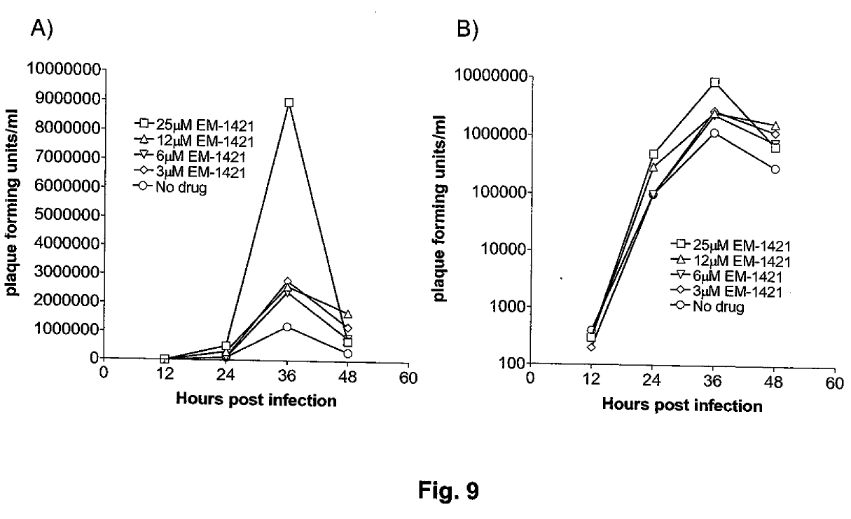

As shown in FIG. 8, where panels A and B display the same data but with linear and log y-axes, respectively, EM-1421 at concentrations tested (3 μM, 6 μM, 12 μM, and 25 μM) did not inhibit A/WS/33 replication in RAW 264.7 macrophages. Instead, EM-1421 enhanced A/WS/33 production from RAW 264.7 macrophages. Again, however, this effect was relatively modest, i.e., less than a 1 log increase (FIG. 8B). Pretreatment of RAW 264.7 cells with EM-1421 further enhanced the growth of A/WS/33. As shown in panels A and B of FIG. 9, the enhancement effect was especially noticeable at the 25 μM concentration of EM-1421 where a 1 log enhancement was measured at the 36 hr time point.

EM-1421 inhibited the growth of influenza strain A/WS/33 with MDCK cells, but enhanced growth with RAW 264.7 macrophages. In both cases, these effects were relatively modest, i.e., appx. 1 log changes in virus titers. Additional viruses and cell types will need to be examined to fully define the effect of EM-1421 on influenza virus replication. In addition, experiments in vivo will be necessary to determine whether these effects significantly affect viral load.

More specifically regarding the method used to determine the effect of M₄N on the production of A/WS/33 from MDCK cells or RAW 264.7 macrophages, the MDCK cells or RAW 264.7 macrophages were inoculated with A/WS/33 at a multiplicity of infection (MOI) of 0.001 or 0.002, respectively. Except for the control where no drug was added, EM-1421 was added to the cells at desired concentrations 30 min after influenza infections were initiated and maintained throughout the experimental period. Culture supernatants were collected at desired time points.

An MDCK-based immunofocus assay was then used to quantitate infectious virus in these supernatants. MDCK cells (5×10⁵/well) were plated in 24 well plates and cultured overnight in virus growth medium which contained: DME media base (#10-013-CV, MediaTech, Herndon Va.) with 10% fetal bovine serum (Atlanta Biologicals, Atlanta Ga.), 25 mM HEPES buffer (#25-060-CL, Mediatech), 1:100 antibiotic/antimycotic solution (#A5955-Sigma-Aldrich, St. Louis Mo.), 1.8 μg/ml bovine serum albumin (#A7906 Sigma-Aldrich), and 2 mg/ml trypsin (#3740, Worthington, Lakewood N.J.). Cells were then washed twice in the same medium without fetal bovine serum. Serial dilutions of virus-containing supernatants were then added for 30 min, followed by an overlay of virus growth medium with 0.6% tragacanth gum (#104792, MP Biomedicals Inc., Solon Ohio). After 24 and 48 hr of incubation the overlay was aspirated, the cells were rinsed with PBS and fixed with 50:50 acetone/methanol. The cells were then stained with anti-HA antibody for focus detection.

To determine the effect of M₄N on the production of A/WS/33 from RAW 264.7 macrophages pretreated with EM-1421, RAW 264.7 macrophages were first incubated with EM-1421 at desired concentrations for 2 hrs. The cells were then inoculated with A/WS/33 at an MOI of 0.002. The EM-1421 was present and maintained in the cell cultures throughout the experimental period. Culture supernatants were collected at desired time points. An MDCK-based immunofocus assay as described above was then used to quantitate infectious virus in these supernatants.

Results from this Example demonstrate that a catecholic butane of the general formula (I) can inhibit the replication of influenza virus in some host cell, indicating that the compound and related catecholic butanes and NDGA derivatives can be used to inhibit the replication or growth of an influenza virus in a host.

### Example

The effects of administering a catecholic butane of the general formula (I), namely M₄N, on the production of TNF-α by RAW 264.7 macrophages infected with influenza strain A/WS/33 were investigated to determine the ability of M₄N to inhibit the induction of TNF-α by influenza infection. Methods similar to that of this Example can be used to determine the effect of any catecholic butane of the general formula (I) on the production of any proinflammatory cytokine in macrophage cells infected with any influenza virus.

Two model systems, a low multiplicity of infection model and a high multiplicity of infection model were used in this study. In the low multiplicity of infection model, infections of RAW 264.7 macrophages were initiated with a very low dose of influenza (MOI=0.002) which then, over the next 24-48 hrs spread throughout the culture. This model attempts to approximate the conditions seen in vivo during a natural infection. However, in this model, infections must be performed in a serum-free infection medium including 2 μg/ml trypsin. It was found that when RAW 264.7 cells were switched from their growth medium (DME with 10% FCS) to the serum-free infection medium, the infection medium itself stimulates the macrophages and elevates background levels of cytokine and lipid mediator production. Trypsin was included in the infection medium because for influenza to spread from cell to cell (in vivo or in vitro) it must be acted upon by extracellular proteases.

In the high MOI model, infections were initiated with a high dose of influenza (MOI=5) which insured that virtually all cells were infected rapidly and synchronously. Strong viral hemagglutinin staining was observed 8 hrs after these infections were initiated. These infections were carried out in normal culture medium which keeps background levels of cytokines and lipid mediators low. However, under these conditions, infectious virions were not produced. The lack of virion production coupled with the high initial dose of influenza means that this model does not approximate influenza infections in vivo.

Therefore, both the low MOI and the high MOI models were used to obtain a more complete view of the effects of influenza and EM-1421 on the metabolism of RAW 264.7.

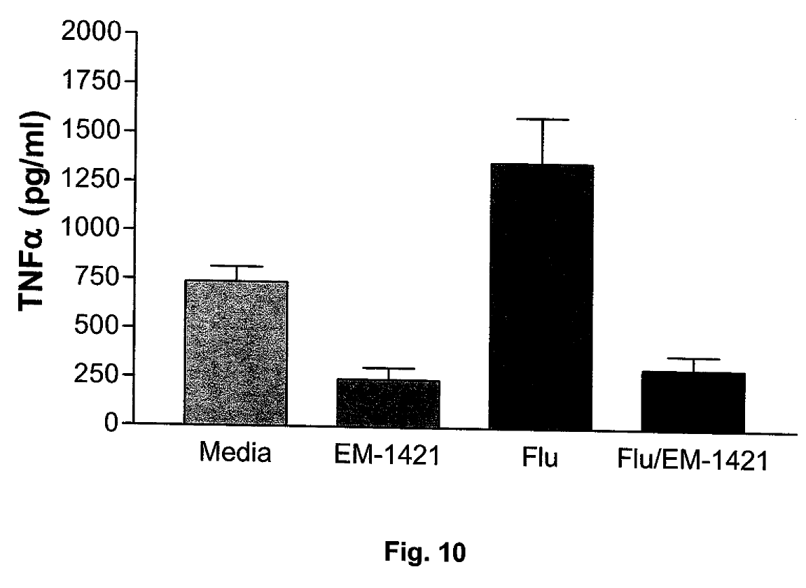

FIGS. 10-12 illustrate the results from a low MOI assay model. In FIG. 10, when RAW 264.7 cells were switched from their growth medium to the serum-free infection medium, the cells produced appx. 750 pg/ml of TNF-α (the “media” bar), which is higher than the 100 pg/ml typically measured when the cells remain in the growth medium. EM-1421 (25 μM) alone reduced this value by about 67%, apparently offsetting the “stress” or “activating” signals associated with the change in media (the “EM-1421” bar). As has been reported, it was found that infection with influenza resulted in an increase in levels of TNF-α (the “Flu” bar). Typically about 80-85% increase in levels of TNF-α was measured after infection with influenza (strain A/WS/33) in this study. EM-1421 (25 μM) completely blocked this influenza-induced increase in the production of TNF-α (the “Flu/EM-1421” bar).

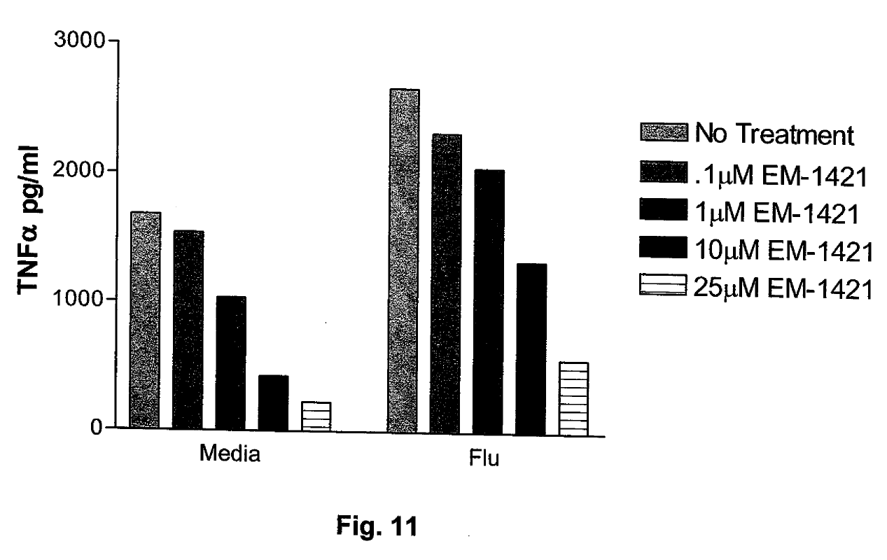

FIG. 11 illustrates the results of a dose response experiment with different concentrations of EM-1421. EM-1421 inhibited the increased production of TNF-α by either the medium alone or the medium and the influenza infection at a final concentration as low as 0.1 μM. Increased concentrations of EM-1421 resulted in increased inhibition.

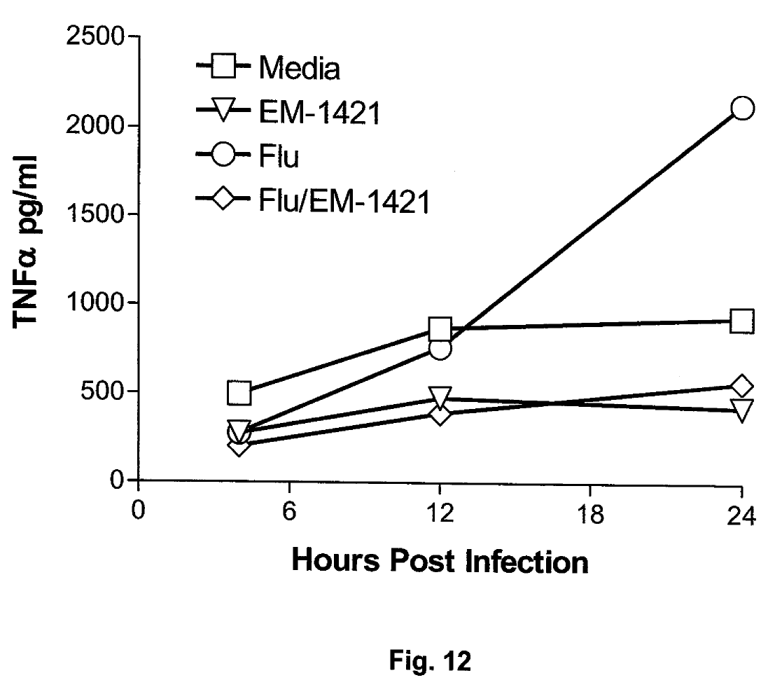

FIG. 12 shows the results of a time course experiment. Cells were incubated with or without the inoculation of influenza strain A/WS/33 (“Flu”), and with or without the treatment of EM-1421 (“EM-1421”). The amount of TNF-α in the culture supernatants was determined at time points indicated in the figure. It was found that the inhibitory effects of EM-1421 appeared to be immediately upon the induction of TNF-α and that the induction of TNF-α remained suppressed throughout the 24 h period.

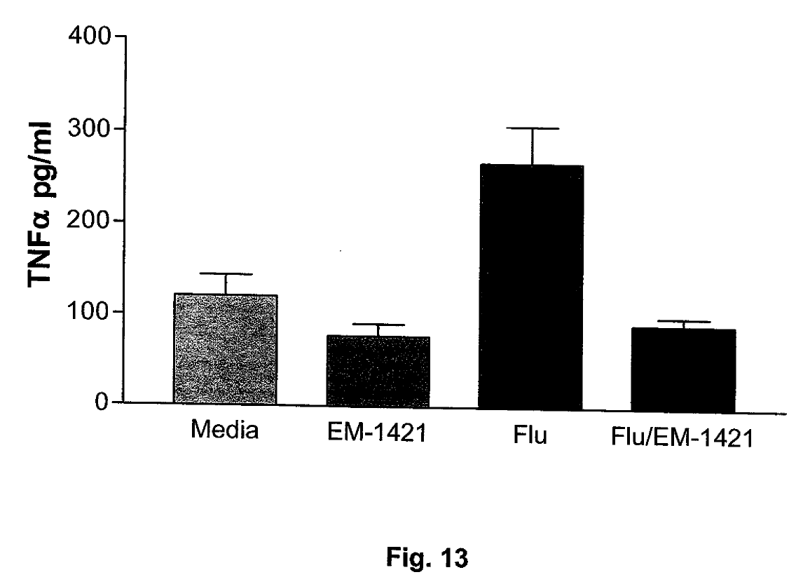

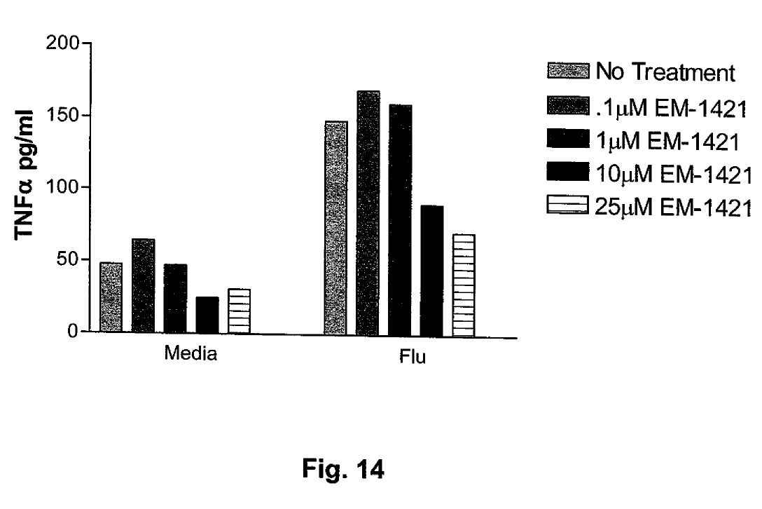

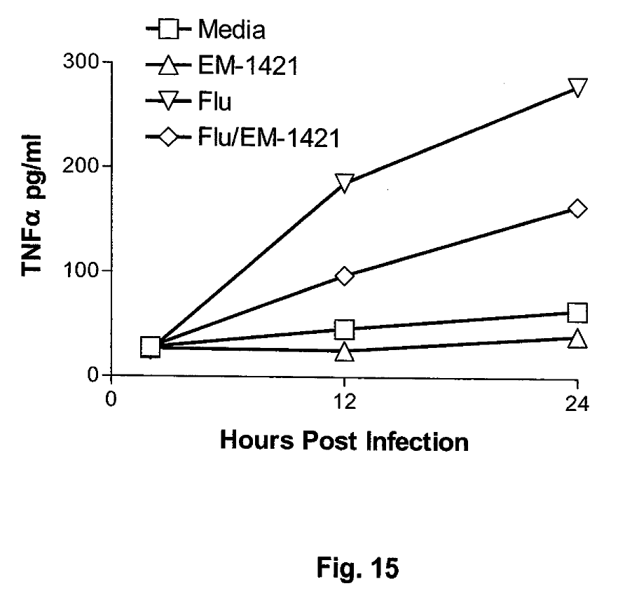

FIGS. 13-15 illustrate the results from a high MOI assay model. RAW 264.7 cells produced appx. 100 pg/ml of TNF-α in the absence of virus infection and the treatment with EM-1421 (the “media” bar). EM-1421 (25 μM) alone reduced this value by about 35% (the “EM-1421” bar). Again, it was found that infection with influenza resulted in an increase in levels of TNF-α, shown as about 135% (the “Flu” bar). EM-1421 (25 μM) again completely blocked this influenza induced increase in the production of TNF-α (the “Flu/EM-1421” bar). FIG. 14 illustrates the results of a dose response experiment. EM-1421, at a final concentration of about 10 μM and 25 μM, inhibited the increased production of TNF-α by influenza infection by about 34% and 60%, respectively. FIG. 15 shows the results of a time course experiment. The inhibitory effects of EM-1421 appeared to be immediately upon the induction of TNF-α and that the induction of TNF-α suppressed by 51% and 55% at 12 and 24 h, respectively.

As shown above, M₄N strongly inhibits the influenza-induced TNF-α overexpression in RAW 264.7 macrophages in both low and high MOI model systems. Thus, M₄N is likely to similarly inhibit the TNF-α response in human macrophages infected with influenza viruses, and particularly the H5N1 influenza subtype. TNF-α is one of the key players in the often lethal inflammatory response in the lung that results from infection with highly virulent H5N1 subtype of influenza. Thus, EM-1421 can dramatically reduce lung inflammation and lethality associated with virulent influenza infection, and ameliorate the severity of the H5N1 disease in humans by controlling cytokine dysregulation. Time course experiments showed that EM-1421 inhibited the induction of TNF-α early in the infection, suggesting that EM-1421 likely acts to inhibit the synthesis and/or release of TNF-α, rather than causing TNF-α degradation.

More specifically regarding the low MOI model used, 1.5×10⁵ RAW 264.7 macrophages cells/well were plated in 24 well plates overnight in DME medium (#D5648, Sigma Aldrich, St. Louis, Mo.) with 10% FCS. The medium was removed and replaced with 200 μl of inoculating virus (strain A/WS/33), at an MOI of 0.002, in virus growth medium (DME base with 2 μg/ml trypsin, 2.5% HEPES buffer, and 0.2% BSA) and allowed the virus to adsorb for 30 min. Then the volume of the medium was increased to 1 ml. EM-1421 was added to a final concentration of 25 μM when the volume was increased to 1 ml. Wells not containing virus and EM-1421 (“media”) or containing EM-1421 only (“EM-1421”) were treated as “mock infected” and received the same manipulations as did infected wells but without the virus. After about 24 hrs incubation, culture supernatants were collected and assayed for TNF-α by ELISA. The data shown are means+/−SEM of two independent experiments with 2 replicate infections performed per experiment. All ELISA points were assayed in duplicate.

In the high MOI model used, 1.5×10⁵ RAW 264.7 macrophages cells/well were plated in 24 well plates overnight in DME medium with 10% FCS. The medium was removed and replaced with 200 μl of inoculating virus (strain A/WS/33), at an MOI of 5, in DME medium with 10% FCS and allowed to adsorb for 30 min. Then the volume of the medium was increased to 1 ml. EM-1421 was added to a final concentration of 25 μM when the volume was increased to 1 ml. Wells not containing virus and EM-1421 (“media”) or containing EM-1421 only (“EM-1421”) were treated as “mock infected” and received the same manipulations as did infected wells but without the virus. After about 24 hrs incubation, culture supernatants were collected and assayed for TNF-α by ELISA. The data shown are means+/−SEM of two independent experiments with 2 replicate infections performed per experiment. All ELISA points were assayed in duplicate.

In the dose response experiments, EM-1421 was added to the medium to a final concentration of 0.1, 1, 10, or 25 μM when the volume of the medium was increased to 1 ml.

In the time course experiments, after about 4, 12, or 24 hrs incubation post inoculating the cell culture with the influenza virus, culture supernatants were collected and assayed for TNF-α by ELISA.

Results from this Example demonstrate that a catecholic butane of the general formula (I) can inhibit the over production of TNF-α in response to influenza viral infection, indicating that the compound and related catecholic butanes and NDGA derivatives can be used to treat diseases or disorders mediated by increased levels of TNF-α upon influenza infection.

### Example 7

The effects of administering a catecholic butane of the general formula (I), namely M₄N, on the production of PGE₂ by RAW 264.7 macrophages infected with influenza strain A/WS/33 were investigated to determine the ability of M₄N to inhibit the induction of PGE₂ by influenza infection. Methods similar to that of this Example can be used to determine the effect of any catecholic butane of the general formula (I) on the production of any pro-inflammatory lipid mediator in macrophage cells infected with any influenza virus.

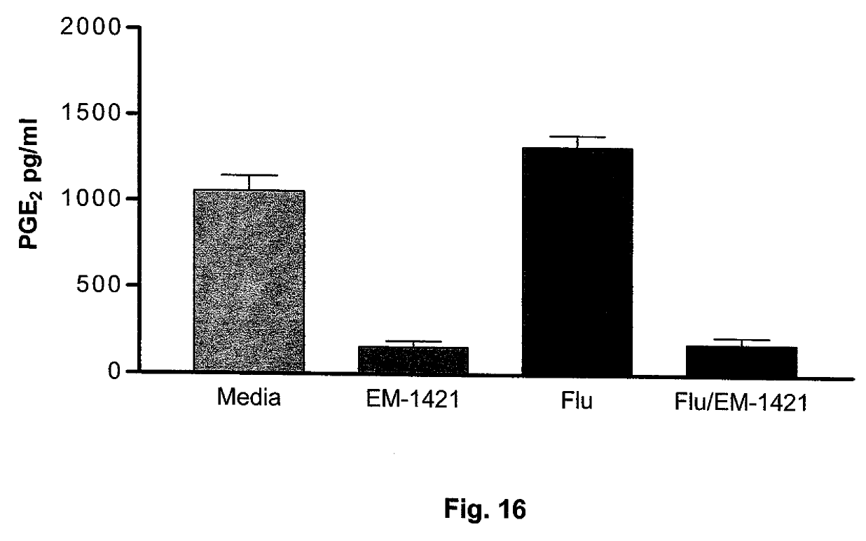

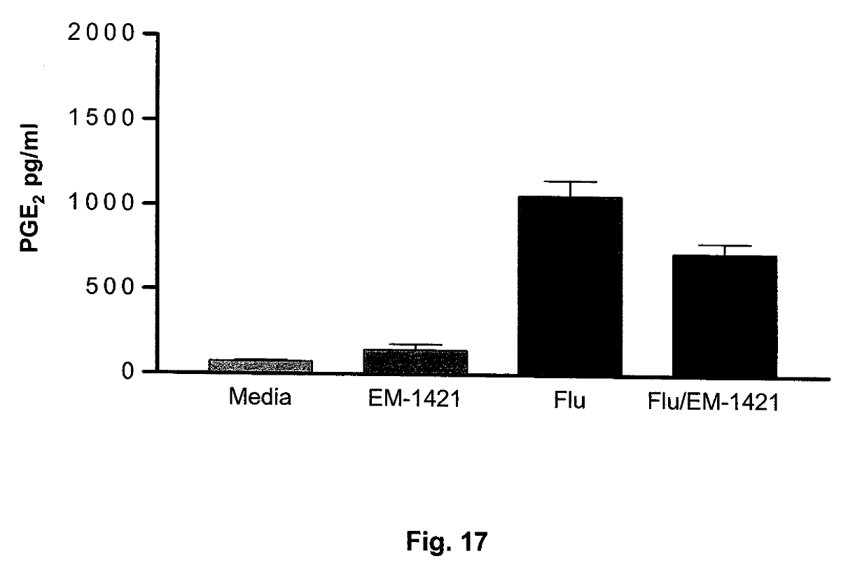

As shown in FIGS. 16 and 17 and explained below, M₄N inhibits the influenza-induced PGE₂ overexpression in RAW 264.7 macrophages. Thus, M₄N is likely to similarly inhibit the TNF-α response in human macrophages infected with influenza viruses, and particularly the H5N1 influenza subtype. Additionally, M₄N may serve to ameliorate the severity of the H5N1 disease in humans by controlling cytokine dysregulation.

Production of PGE₂ during influenza infection has not been studied extensively. Again, both the low MOI and the high MOI models were used in this study.

FIG. 16 illustrates the results from a low MOI assay model. When RAW 264.7 cells were switched from their growth medium to the serum-free infection medium, the cells produced appx. 1 ng/ml of PGE₂ (the “media” bar). EM-1421 (25 μM) alone strongly reduced this value (the “EM-1421” bar). Infection with influenza reproducibly resulted in an increase in levels of PGE₂ by approx. 30% (the “Flu” bar). EM-1421 (25 μM) again strongly blocked this influenza induced increase in the production of PGE₂ (the “Flu/EM-1421” bar).

FIG. 17 illustrates the results from a high MOI assay model. RAW 264.7 cells produced very low level of PGE₂ (appx. 75 pg/ml) in the absence of virus infection and the treatment with EM-1421 (the “media” bar). EM-1421 (25 μM) alone increased the level of PGE₂ by about two fold (the “EM-1421” bar). However, because PGE₂ was produced at low levels in “media” and “EM-1421” wells, it is difficult to predict the significance of these observations. Infection with influenza resulted in a dramatic increase in levels of PGE₂, shown as about 1,300% to about 1,100 pg/ml (the “Flu” bar). EM-1421 (25 μM) reduced this influenza induced increase in the production of PGE₂ by 32% (the “Flu/EM-1421” bar).

The role that lipid mediators play in influenza-induced lung inflammation has not been well characterized. Results obtained in this study revealed new information. In the low MOI model (FIG. 16), the serum-free infection medium resulted in high levels of background PGE₂ production; infection with influenza virus caused additional small and reproducible increase in levels of PGE₂; and EM-1421 completely suppressed the increased production of PGE₅ by both the medium and the influenza infection. Results from the low MOI model are in agreement with the observed strong suppression of LPS-induced production of PGE₂ by EM-1421. In contrast, the high MOI model (FIG. 17) revealed strong induction of PGE₂ (with low background) by influenza infection but only moderate suppression of this induction by EM-1421. Further experiments are to be conducted to explain the different results observed from the low and high MOI model systems.

More specifically regarding the low MOI model used, 1.5×10⁵ RAW 264.7 macrophages cells/well were plated in 24 well plates overnight in DME medium with 10% FCS. The medium was removed and replaced with 200 μl of inoculating virus (strain A/WS/33), at an MOI of 0.002, in virus growth medium (DME base with 2 μg/ml trypsin, 2.5% HEPES buffer, and 0.2% BSA) and allowed the virus to adsorb for 30 min. Then the volume of the medium was increased to 1 ml. EM-1421 was added to a final concentration of 25 μM when the volume was increased to 1 ml. Wells not containing virus and EM-1421 (“media”) or containing EM-1421 only (“EM-1421”) were treated as “mock infected” and received the same manipulations as did infected wells but without the virus. After about 24 hrs incubation, culture supernatants were collected and assayed for PGE₂ by ELISA. The data shown are means+/−SEM of two independent experiments with 2 replicate infections performed per experiment. All ELISA points were assayed in duplicate.

In the high MOI model used, 1.5×10⁵ RAW 264.7 macrophages cells/well were plated in 24 well plates overnight in DME medium with 10% FCS. The medium was removed and replaced with 200 μl of inoculating virus (strain A/WS/33), at an MOI of 5, in DME medium with 10% FCS and allowed to adsorb for 30 min. Then the volume of the medium was increased to 1 ml. EM-1421 was added to a final concentration of 25 μM when the volume was increased to 1 ml. Wells not containing virus and EM-1421 (“media”) or containing EM-1421 only (“EM-1421”) were treated as “mock infected” and received the same manipulations as did infected wells but without the virus. After about 24 hrs incubation, culture supernatants were collected and assayed for PGE₂ by ELISA. The data shown are means+/−SEM of two independent experiments with 2 replicate infections performed per experiment. All ELISA points were assayed in duplicate.

Results from this Example demonstrate that a catecholic butane of the general formula (I) can inhibit the over production of PGE₂ in response to influenza viral infection, indicating that the compound and related catecholic butanes and NDGA derivatives can be used to treat diseases or disorders mediated by increased levels of PGE₂ upon influenza infection.

### Example 8

The effects of administering a catecholic butane of the general formula (I), namely M₄N, on the production of cytokines by RAW 264.7 macrophages were investigated to determine the influenza-induced production of cytokines in addition to TNF-α and the ability of M₄N to inhibit the induced production.

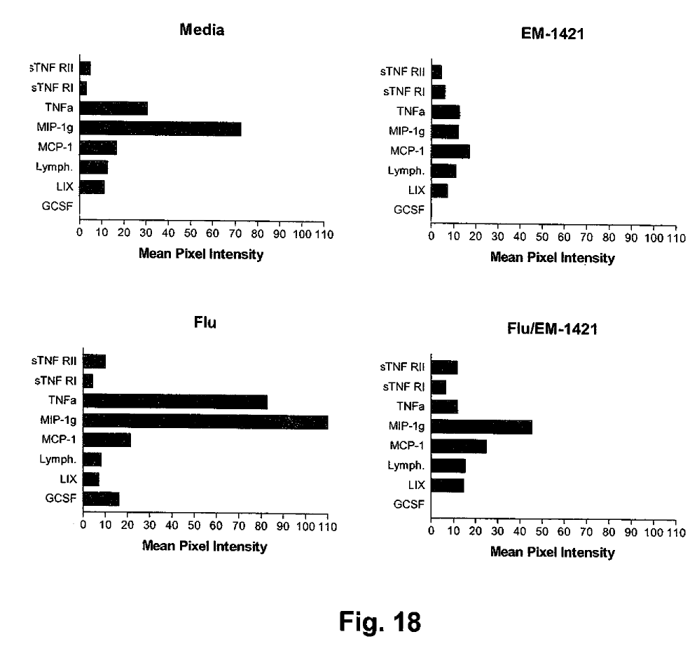

Antibody (“Ab”) array technology was used in this study. As shown in FIG. 18, of the 40 cytokines, chemokines, receptors and proteases on the array, 8 were detected in this experiment. Again, under low MOI conditions, switching RAW 264.7 cells from growth medium to the serum-free infection medium containing trypsin induced the production of substantial levels of TNF-α, high levels of the chemokine MIP-1γ. Flu infection resulted in strong increase in the levels of TNF-α and MIP-1γ, and relatively modest increases in the levels of the sTNFRII and the chemokine MCP-1. Flu infection also induced the production of the cytokine G-CSF which was not detected in the media control sample. EM-1421 (25 μM) blocked many of these effects. The medium induced production of TNF-α and MIP-1γ was completely blocked by EM-1421 as was the flu-induced production of TNF-α. The flu-induced production of MIP-1γ was blocked by appx. 60% and the production of G-CSF was blocked completely. In contrast, EM-1421 did not inhibit the flu-induced production of sTNF RII or MCP-1.

ELISA assays were performed for the cytokines Interferon-β (IFN-β) and IL-6. IFN-β was not included on the array and infections with influenza A can induce this cytokine. However, under both low (A) and high (B) MOI conditions, no significant amount of IFN-β was detected from the culture supernatants of the infected cells 24 h after the inoculation of the virus (data not shown). The ability of influenza A to induce interferon β is highly strain dependent (Hayman, et al. 2006, Virology, 347:52) and apparently strain A/WS/33 is a non-inducer. EM-1421 also did not induce the production of IFN-β.

Although induction of IL-6 has also been reported for certain strains of influenza, no IL-6 induction was detected from the array analysis described supra, suggesting that strain A/WS/33 is also a non-inducer of this cytokine. No significant levels of IL-6 following infection with strain A/WS/33 were detected from the ELISA assay at either low (A) or high (B) MOI, confirming the result of array analysis. Low levels of IL-6 were detected following treatment with EM-1421 under low MOI conditions. However, because the levels of IL-6 were extremely low (10 pg/ml), it is difficult to predict the significance of this observation.

In addition to TNF-α, the array analysis we performed revealed that EM-1421 also blocked the influenza-induced production of MIP-1γ and G-CSF. MIP1-γ, also known as CCL9, is a chemokine whose activity has been linked to a number of cellular processes in vivo including inflammation in the lung (Rosenblum-Lichtenstein, et al., 2006, Am. J. Resp. Cell. Mol. Biol. 35:415). G-CSF is critical for regulating production of neutrophils and mice lacking this gene show reduced levels of neutrophil infiltration into the lung (Gregory, et al., 2006, Blood, epub. ahead of print). Suppression of both these molecules by EM-1421 lends further support that EM-1421 can prevent influenza-associated inflammation in the lung. The A/WS/33 strain of influenza used in these experiments did not induce several of the cytokines and chemokines that have been reported to accompany influenza infection including IFN-β, IL-6, and RANTES. Influenza A strains vary widely in their ability to induce cytokines and chemokines. Experiments are underway with other influenza strain, such as A/PR/8/34, which has been reported to induce a number of cytokines and chemokines in addition to TNF-α (Wareing, et al., 2004, J. Leukoc. Biol. 76:886).

More specifically regarding the method used to determine the effect of M₄N on the production of cytokines, “mouse inflammatory array-1” (RayBiotech, Inc., Atlanta, Ga.) was used in the method. RAW264.7 macrophages were purchased, cultured, stimulated with LPS (1 μg/ml), treated with EM-1421 (25 μM), and harvested in accordance with the procedure set forth in Example 1. Supernatants were collected from RAW 264.7 macrophages cultures (1.5×10⁵ cells/well) 24 h after incubation with either medium (DME base with 2 mg/ml trypsin, 2.5% HEPES buffer, and 0.2% BSA), 0.002 MOI A/WS/33 influenza A, 25 μM EM-1421 or both influenza and EM-1421. Cytokines in the supernatants were measured using the mouse inflammatory array-1 according to the manufacturer's instruction. Briefly, culture supernatants were incubated with nitrocellulose Ab arrays for about 2 h, washed, exposed to secondary Ab solution, developed with ECL solution and exposed to X-ray film. Array autoradiographs were scanned. Photoshop (Adobe) was used to analyze and determine mean pixel intensity for each array position. Mean values for duplicate spots are plotted for the 8 detected products. The other 32 products on the array which were not detected are not shown. Positive control spots were 100-110 units on each array and SEM was less than 5% between duplicate spots. Lymph.=lymphotactin.

More specifically regarding the ELISA method used to detect IFN-β or IL-6 production by RAW 264.7 macrophages, 1.5×10⁵ RAW264.7 macrophage cells/well were plated in 24 well plates overnight in DME media with 10% FCS. Under low MOI assay condition, medium was removed and replaced with 200 μl of inoculating virus (strain A/WS/33) in virus growth media (DME base with 2 μg/ml trypsin, 2.5% HEPES buffer, and 0.2% BSA), at a MOI of 0.002, and allowed to adsorb for 30 min. Under high MOI assay condition, medium was removed and replaced with 200 μl of inoculating virus (strain A/WS/33) in DME medium with 10% FCS and allowed to adsorb for 30 min. In the high MOI model used, 1.5×10⁵ RAW 264.7 macrophages cells/well were plated in 24 well plates overnight in DME medium with 10% FCS. The medium was removed and replaced with 200 μl of inoculating virus (strain A/WS/33), at an MOI of 5, in DME medium with 10% FCS, at a MOI of 5, and allowed to adsorb for 30 min. Then the volume of the medium was increased to 1 ml. EM-1421 was added to a final concentration of 25 μM when the volume was increased to 1 ml. Wells not containing virus and EM-1421 (“media”) or containing EM-1421 only (“EM-1421”) were treated as “mock infected” and received the same manipulations as did infected wells but without the virus. After about 24 hrs incubation, culture supernatants were collected and assayed for IFN-β or IL-6 by ELISA. The data shown are means of two independent experiments with 2 replicate infections performed per experiment. Where not shown, SEM were less than symbol size. ELISA points were assayed in duplicate.

Results from this Example demonstrate that a catecholic butane of the general formula (I) can inhibit the over production of several other cytokines in addition to TNF-α in response to influenza viral infection, indicating that the compound and related catecholic butanes and NDGA derivatives can be used to treat diseases or disorders mediated by increased levels of the cytokines upon influenza infection.

The Examples provided herein demonstrate that a catecholic butane of the general formula (I) can inhibit the overproduction of pro-inflammatory cytokines, such as TNF-α, and the overproduction of pro-inflammatory lipid mediators, such as PGE₂, induced by influenza viral infection, and that a catecholic butane of the general formula (I) can also reduce the TNF-α mediated apoptosis and thus the replication of an influenza virus in a host cell. These results indicate that catecholic butanes are useful for the treatment of influenza viral infections and associated diseases and disorders.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A method of treating an influenza viral infection in a subject, comprising administering to the subject a therapeutically effective amount of a catecholic butane of the general formula (I) or a pharmaceutically acceptable salt thereof: 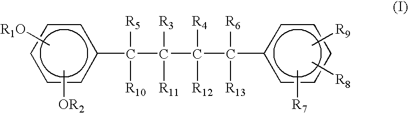
wherein R₁ and R₂ each independently represents a hydrogen, a lower alkyl, a lower acyl, an alkylene, or —OR₁ and —OR₂ each independently represents an unsubstituted or substituted amino acid residue or pharmaceutically acceptable salt thereof; R₃, R₄, R₅, R₆, R₁₀, R₁₁, R₁₂ and R₁₃ each independently represents a hydrogen, or a lower alkyl; and R₇, R₈ and R₉ each independently represents a hydrogen, —OH, a lower alkoxy, a lower acyloxy, an unsubstituted or substituted amino acid residue or salt thereof, or any two adjacent groups together may be an alkyene dioxy; with the proviso that where one of R₇, R₈ and R₉ represents a hydrogen, then —OR₁, —OR₂ and the other two of R₇, R₈ and R₉ do not simultaneously represent —OH.

2. The method according to claim 1, wherein the catecholic butane or the pharmaceutically acceptable salt thereof is administered by one or more routes of administration selected from the group consisting of intranasal administration; oral administration; inhalation administration; subcutaneous administration; transdermal administration; intravenous administration; buccal administration; intraperitoneal administration; intraocular administration; peri-ocular administration; intramuscular administration; implantation administration; infusion; and central venous administration.

3. The method according to claim 1, wherein the catecholic butane or the pharmaceutically acceptable salt thereof is administered orally or intravenously.

4. The method according to claim 1, wherein the catecholic butane or the pharmaceutically acceptable salt thereof is administered in a composition comprising the catecholic butane or the pharmaceutically acceptable salt thereof and a pharmaceutically acceptable carrier or excipient.

5. The method according to claim 4, wherein the pharmaceutically acceptable carrier or excipient comprises: (a) a water-soluble organic solvent; (b) a cyclodextrin or a modified cyclodextrin; (c) an ionic, non-ionic or amphipathic surfactant; (d) a modified cellulose; or (e) a water-insoluble lipid; or a combination of any of the (a)-(e).

6. The method according to claim 4, wherein the pharmaceutically acceptable carrier or excipient comprises at least one or a mixture of dimethyl sulfoxide (DMSO), phosphate buffered saline, saline, a lipid based formulation, a liposomal formulation, a nanoparticle formulation, a micellar formulation, a water soluble formulation, a biodegradable polymer, an aqueous preparation, a hydrophobic preparation, a lipid based vehicle, a polymer formulation, a cyclodextrin, a modified cyclodextrin, a sustained release formulation, a surfactant, a dietary fat, or a dietary oil.

7. The method according to claim 6, wherein the nanoparticle formulation is selected from the group consisting of at least one or a mixture of poly(DL-lactide-co-glycolide), poly vinyl alcohol, d-α-tocopheryl polyethylene glycol 1000 succinate, and poly(lactide-co-glycolide)-monomethoxy-poly(polyethylene glycol).

8. The method according to claim 6, wherein the liposomal formulation is selected from the group consisting of at least one or a mixture of a formulation comprising phosphatidylcholine, cholesterol, PEG-DPPE, distearoylphosphatidylcholine, cholesterol, and PEG-DPPE, and a formulation comprising 1-2-dioleoyl-sn-glycero-3-phosphocholine, 1-2-dipalmitoyl-sn-glycero-3-phospho-rac-(1-glycerol) sodium salt, cholesterol, triolein, and tricaprylin.

9. The method according to claim 6, wherein the pharmaceutically acceptable carrier or excipient comprises at least one or a mixture of a corn oil, castor oil, peanut oil, or a dimethyl sulfoxide.

10. The method according to claim 6, wherein the polymer formulation comprises one ingredient selected from the group consisting of 1,3-bis(p-carboxyphenoxy)propane, sebacic acid, poly(ethylene-co-vinyl acetate), and poly(lactide-co-glycolide).

11. The method according to claim 4, wherein the pharmaceutically acceptable carrier or excipient allows for at least one or a mixture of a high local concentration and a sustained release over a period of time of the catecholic butane or the pharmaceutically acceptable salt thereof.

12. The method according to claim 4, wherein the composition is in a form selected from the group consisting of a powder, an aerosol, a cream, an ointment, a gel, a tablet, a capsule, a pill, a caplet, a granule, a syrup, a solution, an oral rinse, an elixir, an emulsion, a suppository, a suspension, a spray, and drops.

13. The method according to claim 1, wherein the catecholic butane is dissolved in saline, dimethyl sulfoxide, or ethanol, prior to administration.

14. The method according to claim 1, wherein the catecholic butane or the pharmaceutically acceptable salt thereof is administered to the subject in combination with a second agent selected from the group consisting of a second anti-influenza agent, an anti-inflammatory agent, an anti-infective agent, and combinations thereof.

15. The method according to claim 14, wherein the anti-inflammatory agent is selected from the group consisting of a corticosteroid and a non-steroidal anti-inflammatory drug.

16. The method according to claim 14, wherein the anti-infective agent is selected from the group consisting of an antibiotic drug, an alcohol, and povidone.

17. The method according to claim 14, wherein the second anti-influenza agent is selected from the group consisting of a second catecholic butane of the general formula (I) or a pharmaceutically acceptable salt thereof, Amantadine, Oseltamivir, Peramivir, Rimantadine, Zanamivir, and Arbidol.

18. The method according to claim 14, wherein the second agent is administered prior to, substantially contemporaneously with, or after administering of the catecholic butane or the pharmaceutically acceptable salt thereof.

19. The method according to claim 1, wherein R₁ and R₂ are independently —H, a lower alkyl, a lower acyl, or —OR₁ and —OR₂ each independently represents an unsubstituted or substituted amino acid residue or pharmaceutically acceptable salt thereof; R₃, R₄, are independently a lower alkyl; R₅, R₆, R₁₀, R₁₁, R₁₂ and R₁₃ are independently —H; and R₇, R₈ and R₉ are independently —H, —OH, a lower alkoxy, a lower acyloxy, or an unsubstituted or substituted amino acid residue or pharmaceutically acceptable salt thereof; wherein the unsubstituted or substituted amino acid residue is bonded to the aromatic ring at the carboxy terminus.

20. The method according to claim 1, wherein R₁ and R₂ are independently —H, a lower alkyl, a lower acyl, or —OR₁ and —OR₂ each independently represents an unsubstituted or substituted amino acid residue or pharmaceutically acceptable salt thereof; R₃, R₄, are independently a lower alkyl; R₅, R₆, R₇, R₁₀, R₁₁, R₁₂ and R₁₃ are independently —H; and R₈ and R₉ are independently —OH, a lower alkoxy, lower acyloxy, or an unsubstituted or substituted amino acid residue or pharmaceutically acceptable salt thereof; wherein the unsubstituted or substituted amino acid residue is bonded to the aromatic ring at the carboxy terminus.

21. The method according to claim 20, wherein R₁ and R₂ are independently —CH₃ or —(C═O)CH₂N(CH₃)₂ or a pharmaceutically acceptable salt thereof.

22. The method according to claim 20, wherein R₈ and R₉ are independently —OCH₃ or —O(C═O)CH₂N(CH₃)₂ or a pharmaceutically acceptable salt thereof.

23. The method according to claim 20, wherein R₁ and R₂ are independently —CH₃, —(C═O)CH₂N(CH₃)₂ or —(C═O)CH₂N⁺H(CH₃)₂.Cl⁻ and R₈ and R₉ are independently —OCH₃, —O(C═O)CH₂N(CH₃)₂ or —O(C═O)CH₂N⁺H(CH₃)₂.Cl⁻.

24. The method according to claim 20, wherein R₁ and R₂ are independently —H or —CH₃ and R₈ and R₉ are independently —OH or —OCH₃, provided that the catecholic butane is not NDGA.

25. The method according to claim 20, wherein R₁ and R₂ are independently —CH₃ and R₈ and R₉ are independently —OCH₃.

26. The method according to claim 1, wherein the catecholic butane or the pharmaceutically acceptable salt thereof is administered to the subject in an amount selected from the group consisting of about 0.01, about 0.05, about 0.1, about 0.5, about 1.0, about 2.5, about 5.0, about 10, about 15, about 25, about 50, about 100, about 150, about 200, about 250, about 300, about 350, and about 400 mg/kg of body weight per dose.

27. The method according to claim 1, wherein the influenza viral infection is caused by an avian influenza virus.

28. The method according to claim 27, wherein the avian influenza virus is influenza virus subtype H5N1.

29. The method according to claim 1, wherein the subject is a human subject.

30. The method according to claim 1, wherein treating the influenza viral infection comprises interdicting, preventing, palliating, or alleviating a disease or a disorder associated with the influenza viral infection in the subject.

31. The method of claim 30, wherein the disease or the disorder associated with the influenza viral infection is selected from the group consisting of systemic inflammatory response, multiorgan dysfunction, acute respiratory distress syndrome, reactive haemophagocytosis, and lymphopenia.

32. The method according to claim 30, wherein the disease or the disorder associated with the influenza viral infection is asthma, pneumonia, post-influenza encephalitis, bacterial myositis, changes in cardiac electrocardiogram, bronchitis, tuberculosis, carcinoma, rheumatoid arthritis, osteoarthritis, scleroderma, systemic lupus erythematosis, cystic fibrosis, cachexia, generalized muscle weakness disorders, cardiac failure, Parkinsons Disease, amyotrophic lateral sclerosis or Guillain-Barre syndrome.

33. The method according to claim 1, wherein treating the influenza viral infection comprises inhibiting, preventing, or reducing the growth of the influenza virus in the subject.

34. A method of treating an influenza viral infection in a subject, comprising administering to the subject a therapeutically effective amount of a nordihydroguaiaretic acid (NDGA) derivative of the general formula (II) or a pharmaceutically acceptable salt thereof: 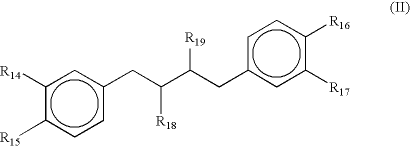
wherein R₁₄, R₁₅, R₁₆ and R₁₇ each independently represents —OH, —OCH₃, —O(C═O)CH₃, or an unsubstituted or substituted amino acid residue or pharmaceutically acceptable salt thereof, R₁₈ and R₁₉ each independently represents —H or a lower alkyl; with the proviso that R₁₄, R₁₅, R₁₆ and R₁₇ are not simultaneously —OH.

35. The method according to claim 34, wherein R₁₄, R₁₅, R₁₆ and R₁₇ each represents —OCH₃.

36. The method according to claim 34, wherein R₁₄, R₁₅, R₁₆ and R₁₇ each represents an unsubstituted or substituted amino acid residue or pharmaceutically acceptable salt thereof, wherein the unsubstituted or substituted amino acid residue is bonded to the aromatic ring at the carboxy terminus.

37. The method according to claim 36, wherein the unsubstituted or substituted amino acid residue comprises unsubstituted or substituted glycinyl acid residue or pharmaceutically acceptable salts thereof.

38. The method according to claim 34, wherein R₁₈ and R₁₉ each independently represents —CH₃ or CH₂CH₃.

39. The method according to claim 34, wherein the nordihydroguaiaretic acid (NDGA) derivative or the pharmaceutically acceptable salt thereof is administered by one or more routes of administration selected from the group consisting of intranasal administration; oral administration; inhalation administration; subcutaneous administration; transdermal administration; intravenous administration; buccal administration; intraperitoneal administration; intraocular administration; peri-ocular administration; intramuscular administration; implantation administration; infusion; and central venous administration.

40. The method according to claim 39, wherein the nordihydroguaiaretic acid derivative or the pharmaceutically acceptable salt thereof is administered orally or intravenously.

41. The method according to claim 34, wherein the nordihydroguaiaretic acid derivative or the pharmaceutically acceptable salt thereof is administered to the subject in an amount selected from the group consisting of about 0.01, about 0.05, about 0.1, about 0.5, about 1.0, about 2.5, about 5.0, about 10, about 15, about 25, about 50, about 100, about 150, about 200, about 250, about 300, about 350, and about 400 mg/kg of body weight per dose.

42. The method according to claim 34, wherein the influenza viral infection is caused by an avian influenza virus.

43. The method according to claim 42, wherein the avian influenza virus is influenza virus subtype H5N1.

44. The method according to claim 34, wherein the subject is a human subject.

45. A method of treating an avian influenza viral infection in a subject, comprising administering to the subject a therapeutically effective amount of a nordihydroguaiaretic acid (NDGA) derivative of the general formula (III) or a pharmaceutically acceptable salt thereof: 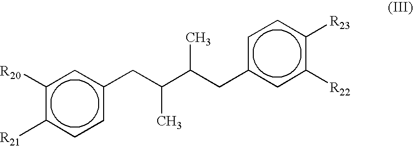
wherein R₂₀, R₂₁, R₂₂ and R₂₃ each independently represents —OH, —OCH₃, —O(C═O)CH₃, or an unsubstituted or substituted amino acid residue or pharmaceutically acceptable salt thereof, with the proviso that R₂₀, R₂₁, R₂₂ and R₂₃ are not simultaneously —OH.

46. The method according to claim 45, wherein R₂₀, R₂₁, R₂₂ and R₂₃ each represents —OCH₃.

47. The method according to claim 45, wherein R20, R21, R22 and R23 each represents an unsubstituted or substituted amino acid residue or pharmaceutically acceptable salt thereof, wherein the unsubstituted or substituted amino acid residue is bonded to the aromatic ring at the carboxy terminus.

48. The method according to claim 47, wherein the unsubstituted or substituted amino acid residue comprises unsubstituted or substituted glycinyl acid residues or pharmaceutically acceptable salts thereof.

49. The method according to claim 45, wherein the nordihydroguaiaretic acid derivative or the pharmaceutically acceptable salt thereof is administered by one or more routes of administration selected from the group consisting of intranasal administration; oral administration; inhalation administration; subcutaneous administration; transdermal administration; intravenous administration; buccal administration; intraperitoneal administration; intraocular administration; peri-ocular administration; intramuscular administration; implantation administration; infusion; and central venous administration.

50. The method according to claim 49, wherein the nordihydroguaiaretic acid derivative or the pharmaceutically acceptable salt thereof is administered orally or intravenously.

51. The method according to claim 45, wherein the nordihydroguaiaretic acid derivative or the pharmaceutically acceptable salt thereof is administered to the subject in an amount selected from the group consisting of about 0.01, about 0.05, about 0.1, about 0.5, about 1.0, about 2.5, about 5.0, about 10, about 15, about 25, about 50, about 100, about 150, about 200, about 250, about 300, about 350, and about 400 mg/kg of body weight per dose.

52. The method according to claim 45, wherein the influenza viral infection is caused by an avian influenza virus.

53. The method according to claim 52, wherein the avian influenza virus is influenza virus subtype H5N1.

54. The method according to claim 45, wherein the subject is a human subject.

55. A method of treating an influenza viral infection in a subject, comprising administering to the subject a therapeutically effective amount of a composition comprising a catecholic butane selected from the group consisting of tri-O-methyl nordihydroguaiaretic acid (NDGA), tetra-O-methyl NDGA, tetra-glycinyl NDGA, tetra-dimethylglycinyl NDGA, or a pharmaceutically acceptable salt thereof, and a pharmaceutically acceptable carrier or excipient.

56. The method according to claim 55, wherein the catecholic butane or the pharmaceutically acceptable salt thereof is administered by one or more routes of administration selected from the group consisting of intranasal administration; oral administration; inhalation administration; subcutaneous administration; transdermal administration; intravenous administration; buccal administration; intraperitoneal administration; intraocular administration; peri-ocular administration; intramuscular administration; implantation administration; infusion; and central venous administration.

57. The method according to claim 56, wherein the catecholic butane or the pharmaceutically acceptable salt thereof is administered orally or intravenously.

58. The method according to claim 55, wherein the catecholic butane or the pharmaceutically acceptable salt thereof is administered to the subject in an amount selected from the group consisting of about 0.01, about 0.05, about 0.1, about 0.5, about 1.0, about 2.5, about 5.0, about 10, about 15, about 25, about 50, about 100, about 150, about 200, about 250, about 300, about 350, and about 400 mg/kg of body weight per dose.

59. The method according to claim 55, wherein the influenza viral infection is caused by an avian influenza virus.

60. The method according to claim 55, wherein the avian influenza virus is influenza virus subtype H5N1.

61. The method according to claim 55, wherein the subject is a human subject.

62. The method according to claim 55, wherein the pharmaceutically acceptable carrier or excipient comprises an oil.

63. The method according to claim 55, wherein the pharmaceutically acceptable carrier or excipient comprises Cremaphor EL, ethanol and saline.

64. The method according to claim 55, wherein the composition comprises at least about 7 mg of the tri-O-methyl NDGA or tetra-O-methyl NDGA per dose.

65. A method of treating a subtype H5N1 influenza viral infection in a human subject, comprising orally administering to the human subject a therapeutically effective amount of a nordihydroguaiaretic acid derivative of the general formula (III) or pharmaceutically acceptable salt thereof, in an amount of about 0.01 to about 400 mg/kg of body weight per dose: 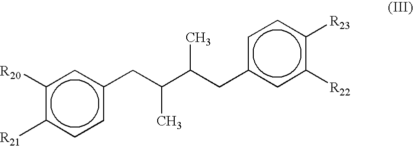
wherein R₂₀, R₂₁, R₂₂ and R₂₃ each represents —OCH₃.

66. The method according to claim 65, wherein treating the subtype H5N1 influenza viral infection comprising interdicting, preventing, palliating, or alleviating a disease or a disorder accompanying the subtype H5N1 influenza viral infection in the human subject.

67. The method of claim 66, wherein the disease or the disorder associated with the subtype H5N1 influenza viral infection in the human subject is selected from the group consisting of systemic inflammatory response, multiorgan dysfunction, acute respiratory distress syndrome, reactive haemophagocytosis, and lymphopenia.

68. The method according to claim 65, wherein the disease or the disorder associated with the subtype H5N1 influenza viral infection in the human subject is asthma, pneumonia, post-influenza encephalitis, bacterial myositis, changes in cardiac electrocardiogram, bronchitis, tuberculosis, carcinoma, rheumatoid arthritis, osteoarthritis, scleroderma, systemic lupus erythematosis, cystic fibrosis, cachexia, generalized muscle weakness disorders, cardiac failure, Parkinsons Disease, amyotrophic lateral sclerosis, or Guillain-Barre syndrome.

69. The method according to claim 65, wherein treating the subtype H5N1 influenza viral infection comprises inhibiting, preventing, or reducing the growth of the subtype H5N1 influenza in the human subject.

70. A method of inhibiting the induction of a proinflammatory cytokine in a cell by an influenza viral infection, comprising administering to the cell an effective amount of a catecholic butane of the general formula I or a pharmaceutically acceptable salt thereof: 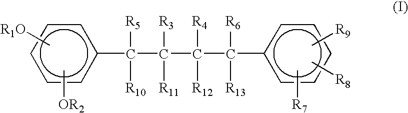
wherein R₁ and R₂ each independently represents a hydrogen, a lower alkyl, a lower acyl, an alkylene, or —OR₁ and —OR₂ each independently represents an unsubstituted or substituted amino acid residue or pharmaceutically acceptable salt thereof; R₃, R₄, R₅, R₆, R₁₀, R₁₁, R₁₂ and R₁₃ each independently represents a hydrogen, or a lower alkyl; and R₇, R₈ and R₉ each independently represents a hydrogen, —OH, a lower alkoxy, a lower acyloxy, an unsubstituted or substituted amino acid residue or salt thereof, or any two adjacent groups together may be an alkyene dioxy.

71. The method according to claim 70, wherein the proinflammatory cytokine is selected from the group consisting of a chemokine, an interleukin (IL), a lymphokine, a tumor necrosis factor (TNF), and an interferon (IFN).

72. The method according to claim 71, wherein the proinflammatory cytokine is selected from the group consisting a TNF-α, a macrophage infectivity potentiator 1γ (MIP-1γ), a granulocyte colony-stimulating factor (G-CSF), an IL-1α, a monocyte chemoattractant protein 1 (MCP-1), an interferon-inducible T-cell alpha chemoattractant (I-TAC), an IL-2, a tissue inhibitor of metalloproteases-1 (TIMP-1), a TIMP-2, a B lymphocyte chemoattractant (BLC), an IL-3, and a regulated upon activation, normal T-cell expressed, and secreted chemokine (RANTES).

73. The method according to claim 72, wherein the pro-inflammatory cytokine is TNF-α.

74. The method according to claim 70, wherein the cell is a macrophage cell.

75. The method according to claim 74, wherein the macrophage cell is a human macrophage cell.

76. The method according to claim 70, wherein the influenza virus is an avian influenza virus.

77. The method according to claim 76, wherein the avian influenza virus is influenza virus subtype H5N1.

78. The method according to claim 70, wherein R₁ and R₂ are independently —H, a lower alkyl, a lower acyl, or —OR₁ and —OR₂ each independently represents an unsubstituted or substituted amino acid residue or pharmaceutically acceptable salt thereof; R₃, R₄, are independently a lower alkyl; R₅, R₆, R₁₀, R₁₁, R₁₂ and R₁₃ are independently —H; and R₇, R₈ and R₉ are independently —H, —OH, a lower alkoxy, a lower acyloxy, or an unsubstituted or substituted amino acid residue or pharmaceutically acceptable salt thereof; wherein the unsubstituted or substituted amino acid residue is bonded to the aromatic ring at the carboxy terminus.

79. The method according to claim 70, wherein R₁ and R₂ are independently —H, a lower alkyl, a lower acyl, or —OR₁ and —OR₂ each independently represents an unsubstituted or substituted amino acid residue or pharmaceutically acceptable salt thereof; R₃, R₄, are independently a lower alkyl; R₅, R₆, R₇, R₁₀, R₁₁, R₁₂ and R₁₃ are independently —H; and R₈ and R₉ are independently —OH, a lower alkoxy, lower acyloxy, or an unsubstituted or substituted amino acid residue or pharmaceutically acceptable salt thereof; wherein the unsubstituted or substituted amino acid residue is bonded to the aromatic ring at the carboxy terminus.

80. The method according to claim 79, wherein R₁ and R₂ are independently —CH₃ or —(C═O)CH₂N(CH₃)₂ or a pharmaceutically acceptable salt thereof.

81. The method according to claim 79, wherein R₈ and R₉ are independently —OCH₃ or —O(C═O)CH₂N(CH₃)₂ or a pharmaceutically acceptable salt thereof.

82. The method according to claim 79, wherein R₁ and R₂ are independently —CH₃, —(C═O)CH₂N(CH₃)₂ or —(C═O)CH₂N⁺H(CH₃)₂.Cl⁻ and R₈ and R₉ are independently —OCH₃, —O(C═O)CH₂N(CH₃)₂ or —O(C═O)CH₂N⁺H(CH₃)₂.Cl⁻.

83. The method according to claim 79, wherein R₁ and R₂ are independently —H or —CH₃ and R₈ and R₉ are independently —OH or —OCH₃.

84. The method according to claim 79, wherein R₁ and R₂ are independently —CH₃ and R₈ and R₉ are independently —OCH₃.

85. A method of inhibiting the induction of a pro-inflammatory lipid mediator in a cell by an influenza viral infection, comprising administering to the cell an effective amount of a catecholic butane of the general formula I or a pharmaceutically acceptable salt thereof: 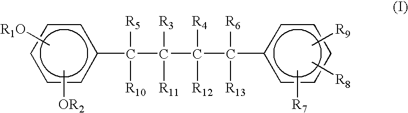
wherein R₁ and R₂ each independently represents a hydrogen, a lower alkyl, a lower acyl, an alkylene, or —OR₁ and —OR₂ each independently represents an unsubstituted or substituted amino acid residue or pharmaceutically acceptable salt thereof; R₃, R₄, R₅, R₆, R₁₀, R₁₁, R₁₂ and R₁₃ each independently represents a hydrogen, or a lower alkyl; and R₇, R₈ and R₉ each independently represents a hydrogen, —OH, a lower alkoxy, a lower acyloxy, an unsubstituted or substituted amino acid residue or salt thereof, or any two adjacent groups together may be an alkyene dioxy.

86. The method according to claim 85, wherein the proinflammatory lipid mediator is prostaglandin or leukotriene.

87. The method according to claim 86, wherein the prostaglandin is selected from the group consisting of prostaglandin E₂ (PGE₂), prostaglandin F_{1α} (PGF^{1α}), prostaglandin F_{2α} (PGF_{2α}), prostaglandin H₂ (PGH₂), and prostacyclin.

88. The method according to claim 87, wherein the prostaglandin is PGE₂.

89. The method according to claim 85, wherein the cell is a macrophage cell.

90. The method according to claim 89, wherein the macrophage cell is a human macrophage cell.

91. The method according to claim 85, wherein the influenza virus is an avian influenza virus.

92. The method according to claim 91, wherein the avian influenza virus is influenza virus subtype H5N1.

93. The method according to claim 85, wherein R₁ and R₂ are independently —H, a lower alkyl, a lower acyl, or —OR₁ and —OR₂ each independently represents an unsubstituted or substituted amino acid residue or pharmaceutically acceptable salt thereof; R₃, R₄, are independently a lower alkyl; R₅, R₆, R₁₀, R₁₁, R₁₂ and R₁₃ are independently —H; and R₇, R₈ and R₉ are independently —H, —OH, a lower alkoxy, a lower acyloxy, or an unsubstituted or substituted amino acid residue or pharmaceutically acceptable salt thereof; wherein the unsubstituted or substituted amino acid residue is bonded to the aromatic ring at the carboxy terminus.

94. The method according to claim 85, wherein R₁ and R₂ are independently —H, a lower alkyl, a lower acyl, or —OR₁ and —OR₂ each independently represents an unsubstituted or substituted amino acid residue or pharmaceutically acceptable salt thereof; R₃, R₄, are independently a lower alkyl; R₅, R₆, R₇, R₁₀, R₁₁, R₁₂ and R₁₃ are independently —H; and R₈ and R₉ are independently —OH, a lower alkoxy, lower acyloxy, or an unsubstituted or substituted amino acid residue or pharmaceutically acceptable salt thereof; wherein the unsubstituted or substituted amino acid residue is bonded to the aromatic ring at the carboxy terminus.

95. The method according to claim 94, wherein R₁ and R₂ are independently —CH₃ or —(C═O)CH₂N(CH₃)₂ or a pharmaceutically acceptable salt thereof.

96. The method according to claim 94, wherein R₈ and R₉ are independently —OCH₃ or —O(C═O)CH₂N(CH₃)₂ or a pharmaceutically acceptable salt thereof.

97. The method according to claim 94, wherein R₁ and R₂ are independently —CH₃, —(C═O)CH₂N(CH₃)₂ or —(C═O)CH₂N⁺H(CH₃)₂.Cl⁻ and R₈ and R₉ are independently —OCH₃, —O(C═O)CH₂N(CH₃)₂ or —O(C═O)CH₂N⁺H(CH₃)₂.Cl⁻.

98. The method according to claim 94, wherein R₁ and R₂ are independently —H or —CH₃ and R₈ and R₉ are independently —OH or —OCH₃.

99. The method according to claim 94, wherein R₁ and R₂ are independently —CH₃ and R₈ and R₉ are independently —OCH₃.

100. A method of inhibiting the induction of tumor necrosis factor alpha (TNF-α) in a macrophage cell by a subtype H5N1 influenza viral infection, comprising administering to the macrophage cell an effective amount of a nordihydroguaiaretic acid derivative of the general formula (III) or pharmaceutically acceptable salt thereof: 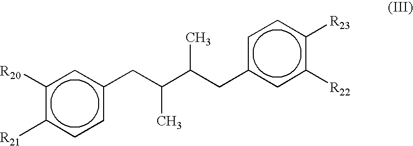
wherein R₂₀, R₂₁, R₂₂ and R₂₃ each represents —OCH₃.

101. A method of inhibiting the induction of prostaglandin E₂ (PGE₂) in a macrophage cell by a subtype H5N1 influenza viral infection, comprising the step of administering to the macrophage cell an effective amount of a nordihydroguaiaretic acid derivative of the general formula (III) or pharmaceutically acceptable salt thereof: 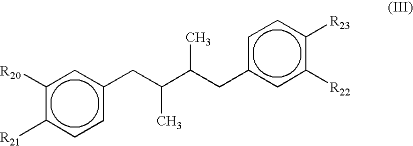
wherein R₂₀, R₂₁, R₂₂ and R₂₃ each represents —OCH₃.

102. A kit comprising a catecholic butane of the general formula I or a pharmaceutically acceptable salt thereof: 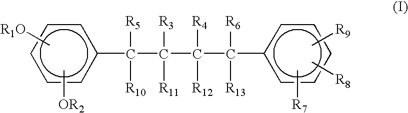
wherein R₁ and R₂ each independently represents a hydrogen, a lower alkyl, a lower acyl, an alkylene, or —OR₁ and —OR₂ each independently represents an unsubstituted or substituted amino acid residue or pharmaceutically acceptable salt thereof; R₃, R₄, R₅, R₆, R₁₀, R₁₁, R₁₂ and R₁₃ each independently represents a hydrogen, or a lower alkyl; and R₇, R₈ and R₉ each independently represents a hydrogen, —OH, a lower alkoxy, a lower acyloxy, an unsubstituted or substituted amino acid residue or salt thereof, or any two adjacent groups together may be an alkyene dioxy; with the proviso that where one of R₇, R₈ and R₉ represents a hydrogen, then —OR₁, —OR₂ and the other two of R₇, R₈ and R₉ do not simultaneously represent —OH; and instructions for treating an influenza viral infection in a subject by using the catecholic butane or the pharmaceutically acceptable salt thereof.

103. The kit according to claim 102, comprising a nordihydroguaiaretic acid (NDGA) derivative of the general formula (II) or a pharmaceutically acceptable salt thereof: 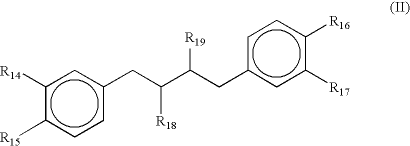
wherein R₁₄, R₁₅, R₁₆ and R₁₇ each independently represents —OH, —OCH₃, —O(C═O)CH₃, or an unsubstituted or substituted amino acid residue or pharmaceutically acceptable salt thereof, R₁₈ and R₁₉ each independently represents —H or a lower alkyl; with the proviso that R₁₄, R₁₅, R₁₆ and R₁₇ are not simultaneously —OH; and instructions for treating an influenza viral infection in a subject by using the NDGA derivative or the pharmaceutically acceptable salt thereof.

104. The kit according to claim 103, wherein the nordihydroguaiaretic acid (NDGA) derivative comprises a NDGA derivative of the general formula (II) or a pharmaceutically acceptable salt thereof: 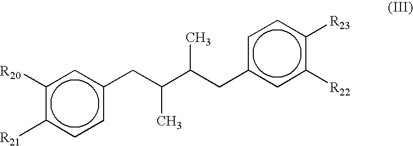
wherein R₂₀, R₂₁, R₂₂ and R₂₃ each independently represents —OH, —OCH₃, —O(C═O)CH₃, or an unsubstituted or substituted amino acid residue or pharmaceutically acceptable salt thereof, with the proviso that R₂₀, R₂₁, R₂₂ and R₂₃ are not simultaneously —OH.

105. The kit according to claim 104, wherein R₂₀, R₂₁, R₂₂ and R₂₃ each represents —OCH₃, and wherein the instructions are for treating a subtype H5N1 influenza viral infection in a human subject.

106. The kit according to claim 102, further comprising a delivery device for administering the catecholic butane or the pharmaceutically acceptable salt thereof to a subject.
